(19) United States
(12) Patent Application Publication
(10) Pub. No.: US 2024/0035073 A1
(43) Pub. Date: Feb. 1, 2024

(54) METHODS FOR DETECTING PROTEINS AND FOR CO-DETECTING PROTEINS AND NUCLEIC ACIDS, AND KITS FOR THE SAME

(71) Applicant: Advanced Cell Diagnostics, Inc., Newark, CA (US)

(72) Inventors: Xiao-Jun Ma, Newark, CA (US); Bingqing Zhang, Newark, CA (US); Henry Lamparski, Newark, CA (US); Courtney Todorov, Newark, CA (US); Lydia Hernandez, Newark, CA (US); Vasudha Murlidhar, Newark, CA (US); Haiyan Wu, Newark, CA (US); HaYeun Ji, Newark, CA (US); Emerald Doolittle, Newark, CA (US); Maithreyan Srinivasan, Newark, CA (US); Li-chong Wang, Newark, CA (US)

(21) Appl. No.: 18/360,414

(22) Filed: Jul. 27, 2023

Related U.S. Application Data
(63) Continuation of application No. PCT/US2023/060962, filed on Jan. 20, 2023.
(60) Provisional application No. 63/301,711, filed on Jan. 21, 2022.

(51) Int. Cl. C12Q 1/6841

(52) U.S. Cl. CPC C12Q 1/6841

(57) ABSTRACT

Disclosed herein are methods for detecting a target protein in a sample and methods for detecting a target protein and a target nucleic acid in a sample. Kits for carrying out the methods are also disclosed.

### CROSS-REFERENCE TO RELATED APPLICATION

This application is a continuation of International Application No. PCT/US2023/060962, filed Jan. 20, 2023, which claims priority to and the benefit of U.S. Provisional Patent Application No. 63/301,711, filed Jan. 21, 2022, each of which are incorporated herein by reference in its entirety.

### FIELD

Embodiments of the present disclosure include methods for detecting a target protein in a biological sample, and methods for detecting a target protein and a target nucleic acid in the same biological sample. Kits for carrying out the methods are also provided.

### BACKGROUND

In situ hybridization (ISH) is a molecular biology technique widely used to detect and localize specific sequences within cells or tissue sections while preserving the cellular and tissue context. ISH, therefore, enables spatial-temporal visualization as well as quantification of gene expression within cells and tissues, which has useful applications in research and in diagnostics. See Hu et al., Biomarker Research 2(1): 1-13 (2014); Ratan et al., Cureus 9(6): e1325 (2017); Weier et al., Expert Review of Molecular Diagnostics 2(2): 109-119 (2002).

Immunohistochemistry (IHC) and immunocytochemistry (ICC) are also powerful techniques that are used to detect and localize specified proteins within tissue sections and cells, while similarly maintaining spatial resolution and cytological context. Similar to ISH, IHC and ICC have broad and complementary applications in research and diagnostics. See Shi et al., Journal of Histochemistry & Cytochemistry 59(11): 13-32 (2011). For example, both approaches provide researchers insights regarding cell identities and states.

Complete characterization of complex cellular interactions within a tissue or cell requires a multi-omic strategy. For example, detecting and analyzing transcriptomic and proteomic information is informative in interrogating complex tissues and in revealing cell-type specific gene expression (see Vanlandewijck et al., Nature 554(7693): 475-482 (2018); Stempl et al., the Journal of Molecular Diagnostics 14(1): 22-29 (2014)), in identifying the cellular sources of secreted proteins (see Liou et al., Cell Reports 19(7): 1322-1333 (2017)), and in visualizing the spatial organization of the various cell types and their interactions. To characterize cells and tissues fully and accurately, both nucleic acids and proteins need to be detected in the same tissue sample, e.g., simultaneously, in a spatially resolved manner.

Despite the critical roles of dual detection, it has been problematic to obtain high-quality signals for both nucleic acids and proteins in the same sample. A significant demand therefore exists for new methods that can detect both signals without interference from each other. The present disclosure addresses these and other needs.

### SUMMARY

In one aspect, provided herein is a method for detecting a target protein in a biological sample, the method comprising (i) contacting the sample with an antibody or a fragment thereof, wherein the antibody or the fragment thereof is covalently attached to an oligonucleotide; (ii) contacting the sample with a signal-generating complex, wherein the signal-generating complex comprises a nucleic acid component capable of hybridizing to the oligonucleotide; and (iii) detecting a signal from the signal-generating complex.

In some embodiments, the antibody or fragment thereof is selected from a monoclonal antibody, a multispecific antibody, a bispecific antibody, a trispecific antibody, a tetravalent antibody, a single-domain antibody, and a chimeric antibody. In some embodiments, step (i) comprises contacting the sample with a composition comprising polyclonal antibodies, wherein a plurality of antibodies in the composition are covalently attached to the oligonucleotide. In some embodiments, the antibody or fragment thereof is selected from a Fab, a scFv, a Fv, a scFv-Fc, a Fab′, a Fab′-SH, a F(ab′)₂, a diabody, a minibody, a tribody, a nanobody, and an affibody.

In some embodiments, the oligonucleotide has a length of about 10 to about 100 nucleotides.

In some embodiments, the oligonucleotide is covalently attached to the antibody via a linker.

In some embodiments, the linker comprises an oligonucleotide sequence.

In some embodiments, step (i) comprises contacting the sample with the antibody or the fragment thereof for about 10 minutes to about 48 hours. In some embodiments, step (i) comprises contacting the sample with the antibody or the fragment thereof at about 4° C. to about 75° C. In some embodiments, step (i) comprises contacting the sample with the antibody or the fragment thereof at room temperature.

In some embodiments, the method further comprises contacting the sample with a blocking agent prior to step (i). In some embodiments, the blocking agent comprises RNA or DNA. In some embodiments, the blocking agent comprises DNA selected from salmon sperm DNA, herring DNA, and calf thymus DNA. In some embodiments, the blocking agent comprises tRNA. In some embodiments, the blocking agent comprises a protein. In some embodiments, the blocking agent is selected from bovine serum albumin, casein, normal goat serum, normal swine serum, normal chicken serum, fish serum, and a plant protein based blocking agent.

In some embodiments, the method further comprises contacting the sample with a crosslinking agent after step (i) and before step (ii). In some embodiments, the crosslinking agent is a fixative. In some embodiments, the fixative is neutral buffered formalin, glutaraldehyde, or glyoxal. In some embodiments, the crosslinking agent is a bis(succinimidyl) polyethylene glycol.

In some embodiments, the method comprises contacting the sample with the crosslinking agent for about 5 minutes to about 24 hours, at a temperature of about 4° C. to about 60° C.

In some embodiments, the method comprises contacting the sample with a protease after the crosslinking step and before step (ii).

In some embodiments, the signal-generating complex comprises: a pre-pre-amplifier, a pre-amplifier, and/or an amplifier; and one or more label probes, wherein each label probe comprises a detectable label. In some embodiments, the signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the biological sample is a tissue specimen or is derived from a tissue specimen. In some embodiments, the biological sample is a blood sample or is derived from a blood sample. In some embodiments, the biological sample is a cytological sample or is derived from a cytological sample. In some embodiments, the biological sample is cultured cells or a sample containing exosomes.

In some embodiments, the antibody or the fragment thereof binds directly to the target protein.

In some embodiments, the method comprises detecting two or more proteins in the sample. In some embodiments, the method comprises (i) contacting the sample with a plurality of antibodies or fragments thereof, wherein antibody or fragment thereof specifically hybridizes to a target protein, and wherein each antibody or fragment thereof is covalently attached to an oligonucleotide; (ii) contacting the sample with a set of pre-amplifiers, wherein the set of pre-amplifiers comprises a plurality of pre-amplifiers, wherein the plurality of pre-amplifiers comprises a pre-amplifier specific for each oligonucleotide, wherein each pre-amplifier comprises binding sites for the oligonucleotide and a plurality of binding sites for an amplifier; (iii) contacting the sample with a set of amplifiers, wherein the set of amplifiers comprises a plurality of subsets of amplifiers specific for each pre-amplifier, wherein each subset of amplifiers comprises a plurality of amplifiers, wherein the amplifiers of a subset of amplifiers comprise a binding site for one of the pre-amplifiers specific for an oligonucleotide and a plurality of binding sites for a label probe; (iv) contacting the sample with a first set of label probes, wherein the first set of label probes comprises a plurality of first subsets of label probes, wherein each subset of label probes is specific for the amplifiers of one of the subsets of amplifiers, wherein each subset of label probes comprises a plurality of label probes, wherein the label probes in each of the subsets of label probes comprise a label and a binding site for the amplifiers of one of the subsets of amplifiers, wherein the labels in each first subset of label probes are distinguishable between the first subsets of label probes and wherein the labels are cleavable, and wherein the first set of label probes specifically label a first subset of target proteins bound to the plurality of antibodies or fragments thereof; (v) detecting the label probes of the first set of label probes bound to the target proteins, thereby detecting the first subset of target proteins; (vi) cleaving the labels from the first set of label probes bound to the first subset of target proteins; (vii) contacting the sample with a second set of label probes, wherein the second set of label probes comprises a plurality of second subsets of label probes, wherein each subset of label probes is specific for the amplifiers of one of the subsets of amplifiers, wherein the second subsets of label probes are specific for amplifiers of different subsets of amplifiers than the first subsets of label probes, wherein each subset of label probes comprises a plurality of label probes, wherein the label probes of each of the subsets of label probes comprise a label and a binding site for the amplifiers of one of the subsets of amplifiers, wherein the labels in each second subset of label probes are distinguishable between the second subsets of label probes and wherein the labels are cleavable, and wherein the second set of label probes specifically label a second subset of target proteins bound to the plurality of antibodies or fragments thereof that is different than the first subset of target proteins; and (viii) detecting the label probes of the second set of label probes bound to the target proteins, thereby detecting the second subset of target proteins, wherein a plurality of target proteins are detected.

In some embodiments, the method further comprises (ix) cleaving the labels from the second set of label probes bound to the second set of target proteins; (x) contacting the sample with a third set of label probes, wherein the third set of label probes comprises a plurality of third subsets of label probes, wherein each subset of label probes is specific for the amplifiers of one of the subsets of amplifiers, wherein the third subsets of label probes are specific for amplifiers of different subsets of amplifiers than the first and second subsets of label probes, wherein each subset of label probes comprises a plurality of label probes, wherein the label probes of each of the subsets of label probes comprise a label and a binding site for the amplifiers of one of the subsets of amplifiers, wherein the labels in each third subset of label probes are distinguishable between the third subsets of label probes and wherein the labels are cleavable, and wherein the third set of label probes specifically label a third subset of target proteins bound to the plurality of antibodies or fragments thereof that is different than the first and second subsets of target proteins; and (xi) detecting the label probes of the third set of label probes bound to the target nucleic acids, thereby detecting the third subset of target nucleic acids.

In another aspect, provided herein is a method for detecting a target protein and a target nucleic acid in a biological sample, the method comprising (i) contacting the sample with an antibody or a fragment thereof, wherein the antibody or the fragment thereof is covalently attached to an oligonucleotide; (ii) contacting the sample with a first signal-generating complex, wherein the first signal-generating complex comprises a nucleic acid component capable of hybridizing to the oligonucleotide; (iii) detecting a signal from the first signal-generating complex to thereby detect the target protein in the sample; and (iv) detecting the target nucleic acid by in situ hybridization.

In some embodiments, the target protein and the target nucleic acid are simultaneously detected in the sample.

In some embodiments, the antibody or fragment thereof is selected from a monoclonal antibody, a multispecific antibody, a bispecific antibody, a trispecific antibody, a tetravalent antibody, a single-domain antibody, and a chimeric antibody. In some embodiments, step (i) comprises contacting the sample with a composition comprising polyclonal antibodies, wherein a plurality of antibodies in the composition are covalently attached to the oligonucleotide. In some embodiments, the antibody or fragment thereof is selected from a Fab, a scFv, a Fv, a scFv-Fc, a Fab′, a Fab′-SH, a F(ab′)₂, a diabody, a minibody, a tribody, a nanobody, and an affibody.

In some embodiments, the oligonucleotide has a length of about 10 to about 100 nucleotides. In some embodiments, the oligonucleotide is covalently attached to the antibody via a linker. In some embodiments, the linker comprises an oligonucleotide sequence.

In some embodiments, step (i) comprises contacting the sample with the antibody or the fragment thereof for about 10 minutes to about 48 hours. In some embodiments, step (i) comprises contacting the sample with the antibody or the fragment thereof at about 4° C. to about 75° C. In some embodiments, step (i) comprises contacting the sample with the antibody or the fragment thereof at room temperature.

In some embodiments, the method further comprises contacting the sample with a blocking agent prior to step (i). In some embodiments, the blocking agent comprises RNA or DNA. In some embodiments, the blocking agent comprises DNA selected from salmon sperm DNA, herring DNA, or calf thymus DNA. In some embodiments, the blocking agent comprises tRNA. In some embodiments, the blocking agent comprises a protein. In some embodiments, the blocking agent is selected from bovine serum albumin, casein, normal goat serum, normal swine serum, normal chicken serum, fish serum, and a plant protein based blocking agent.

In some embodiments, the method further comprises contacting the sample with a crosslinking agent after step (i) and before step (ii). In some embodiments, the crosslinking agent is a fixative. In some embodiments, the fixative is neutral buffered formalin. In some embodiments, the crosslinking agent is a bis(succinimidyl) polyethylene glycol.

In some embodiments, the method comprises contacting the sample with the crosslinking agent for about 5 minutes to about 24 hours, at a temperature of about 4° C. to about 60° C. In some embodiments, the method comprises contacting the sample with a protease after the crosslinking step and before step (ii).

In some embodiments, the first signal-generating complex comprises a pre-pre-amplifier, a pre-amplifier, and/or an amplifier; and one or more label probes, wherein each label probe comprises a detectable label.

In some embodiments, the first signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the antibody or the fragment thereof binds directly to the target protein.

In some embodiments, the method comprises detecting two or more proteins in the sample.

In some embodiments, the method comprises (i) contacting the sample with a plurality of antibodies or fragments thereof, wherein antibody or fragment thereof specifically hybridizes to a target protein, and wherein each antibody or fragment thereof is covalently attached to an oligonucleotide; (ii) contacting the sample with a set of pre-amplifiers, wherein the set of pre-amplifiers comprises a plurality of pre-amplifiers, wherein the plurality of pre-amplifiers comprises a pre-amplifier specific for each oligonucleotide, wherein each pre-amplifier comprises binding sites for the oligonucleotide and a plurality of binding sites for an amplifier; (iii) contacting the sample with a set of amplifiers, wherein the set of amplifiers comprises a plurality of subsets of amplifiers specific for each pre-amplifier, wherein each subset of amplifiers comprises a plurality of amplifiers, wherein the amplifiers of a subset of amplifiers comprise a binding site for one of the pre-amplifiers specific for an oligonucleotide and a plurality of binding sites for a label probe; (iv) contacting the sample with a first set of label probes, wherein the first set of label probes comprises a plurality of first subsets of label probes, wherein each subset of label probes is specific for the amplifiers of one of the subsets of amplifiers, wherein each subset of label probes comprises a plurality of label probes, wherein the label probes in each of the subsets of label probes comprise a label and a binding site for the amplifiers of one of the subsets of amplifiers, wherein the labels in each first subset of label probes are distinguishable between the first subsets of label probes and wherein the labels are cleavable, and wherein the first set of label probes specifically label a first subset of target proteins bound to the plurality of antibodies or fragments thereof; (v) detecting the label probes of the first set of label probes bound to the target proteins, thereby detecting the first subset of target proteins; (vi) cleaving the labels from the first set of label probes bound to the first subset of target proteins; (vii) contacting the sample with a second set of label probes, wherein the second set of label probes comprises a plurality of second subsets of label probes, wherein each subset of label probes is specific for the amplifiers of one of the subsets of amplifiers, wherein the second subsets of label probes are specific for amplifiers of different subsets of amplifiers than the first subsets of label probes, wherein each subset of label probes comprises a plurality of label probes, wherein the label probes of each of the subsets of label probes comprise a label and a binding site for the amplifiers of one of the subsets of amplifiers, wherein the labels in each second subset of label probes are distinguishable between the second subsets of label probes and wherein the labels are cleavable, and wherein the second set of label probes specifically label a second subset of target proteins bound to the plurality of antibodies or fragments thereof that is different than the first subset of target proteins; and (viii) detecting the label probes of the second set of label probes bound to the target proteins, thereby detecting the second subset of target proteins, wherein a plurality of target proteins are detected.

In some embodiments, the method further comprises (ix) cleaving the labels from the second set of label probes bound to the second set of target proteins; (x) contacting the sample with a third set of label probes, wherein the third set of label probes comprises a plurality of third subsets of label probes, wherein each subset of label probes is specific for the amplifiers of one of the subsets of amplifiers, wherein the third subsets of label probes are specific for amplifiers of different subsets of amplifiers than the first and second subsets of label probes, wherein each subset of label probes comprises a plurality of label probes, wherein the label probes of each of the subsets of label probes comprise a label and a binding site for the amplifiers of one of the subsets of amplifiers, wherein the labels in each third subset of label probes are distinguishable between the third subsets of label probes and wherein the labels are cleavable, and wherein the third set of label probes specifically label a third subset of target proteins bound to the plurality of antibodies or fragments thereof that is different than the first and second subsets of target proteins; and (xi) detecting the label probes of the third set of label probes bound to the target nucleic acids, thereby detecting the third subset of target nucleic acids.

In some embodiments, the target nucleic acid is RNA. In some embodiments, the RNA is selected from messenger RNA (mRNA), microRNA (miRNA), a pre-miRNA, ribosomal RNA (rRNA), mitochondrial RNA, non-coding RNA, a circular RNA, a small interfering RNA (siRNA), a guide RNA, or an antisense oligonucleotide. In some embodiments, the target nucleic acid is DNA.

In some embodiments, the step of detecting the target nucleic acid by in situ hybridization comprises (i) providing one or more target probe(s) capable of hybridizing to the target nucleic acid; (ii) providing a second signal-generating complex capable of hybridizing to the one or more target probe(s), wherein the second signal-generating complex comprises a nucleic acid component capable of hybridizing to the one or more target probe(s); (iii) hybridizing the target nucleic acid to the one or more target probe(s); and (iv) capturing the second signal-generating complex to the one or more target probe(s) and thereby capturing the second signal-generating complex to the target nucleic acid.

In some embodiments, each of the one or more target probe(s) comprises a target (T) section and a label (L) section, wherein the T section is a nucleic acid sequence complementary to a section on the target nucleic acid and the L section is a nucleic acid sequence complementary to a section on the nucleic acid component of the second signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the second signal-generating complex.

In some embodiments, the second signal-generating complex comprises: a pre-pre-amplifier, a pre-amplifier, and/or an amplifier; and one or more label probes, wherein each label probe of the second signal-generating complex comprises a detectable label that is distinct from the detectable label of the first signal-generating complex. In some embodiments, the second signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the biological sample is a tissue specimen or is derived from a tissue specimen. In some embodiments, the biological sample is a blood sample or is derived from a blood sample. In some embodiments, the biological sample is a cytological sample or is derived from a cytological sample. In some embodiments, the biological sample is cultured cells or a sample containing exosomes.

In some embodiments, the method comprises detecting two or more nucleic acids in the sample.

In some embodiments, the method comprises (i) contacting the sample with a plurality of target probe sets, wherein each target probe set comprises a pair of target probes that specifically hybridize to a target nucleic acid; (ii) contacting the sample with a set of pre-amplifiers, wherein the set of pre-amplifiers comprises a plurality of pre-amplifiers, wherein the plurality of pre-amplifiers comprises a pre-amplifier specific for each target probe set, wherein each pre-amplifier comprises binding sites for the pair of target probes of one of the target probe sets and a plurality of binding sites for an amplifier; (iii) contacting the sample with a set of amplifiers, wherein the set of amplifiers comprises a plurality of subsets of amplifiers specific for each pre-amplifier, wherein each subset of amplifiers comprises a plurality of amplifiers, wherein the amplifiers of a subset of amplifiers comprise a binding site for one of the pre-amplifiers specific for a target probe set and a plurality of binding sites for a label probe; (iv) contacting the sample with a first set of label probes, wherein the first set of label probes comprises a plurality of first subsets of label probes, wherein each subset of label probes is specific for the amplifiers of one of the subsets of amplifiers, wherein each subset of label probes comprises a plurality of label probes, wherein the label probes in each of the subsets of label probes comprise a label and a binding site for the amplifiers of one of the subsets of amplifiers, wherein the labels in each first subset of label probes are distinguishable between the first subsets of label probes and wherein the labels are cleavable, and wherein the first set of label probes specifically label a first subset of target nucleic acids hybridized to the plurality of target probe sets; (v) detecting the label probes of the first set of label probes bound to the target nucleic acids, thereby detecting the first subset of target nucleic acids; (vi) cleaving the labels from the first set of label probes bound to the first subset of target nucleic acids; (vii) contacting the sample with a second set of label probes, wherein the second set of label probes comprises a plurality of second subsets of label probes, wherein each subset of label probes is specific for the amplifiers of one of the subsets of amplifiers, wherein the second subsets of label probes are specific for amplifiers of different subsets of amplifiers than the first subsets of label probes, wherein each subset of label probes comprises a plurality of label probes, wherein the label probes of each of the subsets of label probes comprise a label and a binding site for the amplifiers of one of the subsets of amplifiers, wherein the labels in each second subset of label probes are distinguishable between the second subsets of label probes and wherein the labels are cleavable, and wherein the second set of label probes specifically label a second subset of target nucleic acids hybridized to the plurality of target probe sets that is different than the first subset of target nucleic acids; and (viii) detecting the label probes of the second set of label probes bound to the target nucleic acids, thereby detecting the second subset of target nucleic acids, wherein a plurality of target nucleic acids are detected.

In some embodiments, the method further comprises (ix) cleaving the labels from the second set of label probes bound to the second set of target nucleic acids; (x) contacting the sample with a third set of label probes, wherein the third set of label probes comprises a plurality of third subsets of label probes, wherein each subset of label probes is specific for the amplifiers of one of the subsets of amplifiers, wherein the third subsets of label probes are specific for amplifiers of different subsets of amplifiers than the first and second subsets of label probes, wherein each subset of label probes comprises a plurality of label probes, wherein the label probes of each of the subsets of label probes comprise a label and a binding site for the amplifiers of one of the subsets of amplifiers, wherein the labels in each third subset of label probes are distinguishable between the third subsets of label probes and wherein the labels are cleavable, and wherein the third set of label probes specifically label a third subset of target nucleic acids hybridized to the plurality of target probe sets that is different than the first and second subsets of target nucleic acids; and (xi) detecting the label probes of the third set of label probes bound to the target nucleic acids, thereby detecting the third subset of target nucleic acids.

In another aspect, provided herein is a kit for detecting a target protein in a biological sample, comprising (i) an antibody or a fragment thereof, wherein the antibody or the fragment thereof is covalently attached to an oligonucleotide; and (ii) a signal-generating complex, wherein the signal-generating complex comprises a nucleic acid component capable of hybridizing to the oligonucleotide.

In some embodiments, the antibody or fragment thereof is selected from a monoclonal antibody, a multispecific antibody, a bispecific antibody, a trispecific antibody, a tetravalent antibody, a single-domain antibody, and a chimeric antibody. In some embodiments, the kit comprises a composition of polyclonal antibodies, wherein a plurality of antibodies in the composition are covalently attached to the oligonucleotide. In some embodiments, the antibody or fragment thereof is selected from a Fab, a scFv, a Fv, a scFv-Fc, a Fab′, a Fab′-SH, a F(ab′)₂, a diabody, a minibody, a tribody, a nanobody, and an affibody.

In some embodiments, the oligonucleotide has a length of about 10 to about 100 nucleotides. In some embodiments, the oligonucleotide is covalently attached to the antibody via a linker. In some embodiments, the linker comprises an oligonucleotide sequence.

In some embodiments, the signal-generating complex comprises: a pre-pre-amplifier, a pre-amplifier, and/or an amplifier; and one or more label probes, wherein each label probe comprises a detectable label. In some embodiments, the signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the antibody or the fragment thereof binds directly to the target protein.

In some embodiments, the kit further comprises a blocking agent, a crosslinking agent, a protease, or any combination thereof In some embodiments, the kit further comprises instructions for carrying out a method of detecting the target protein in the biological sample.

In some embodiments, the kit is for detecting two or more target proteins in the biological sample, and the kit comprises (i) a plurality of antibodies or fragments thereof, wherein each antibody or fragment thereof is covalently attached to a different oligonucleotide; and (ii) a plurality of signal-generating complexes, wherein each signal-generating complex comprises a nucleic acid component capable of hybridizing to one of the oligonucleotides.

In another aspect, provided herein is a kit for detecting a target protein in a biological sample, comprising (i) an oligonucleotide comprising a reactive moiety for conjugation to an antibody; and (ii) a signal-generating complex, wherein the signal-generating complex comprises a nucleic acid component capable of hybridizing to the oligonucleotide.

In some embodiments, the oligonucleotide has a length of about 10 to about 100 nucleotides.

In some embodiments, the kit further comprises a crosslinker for conjugation of the oligonucleotide to the antibody.

In some embodiments, the signal-generating complex comprises: a pre-pre-amplifier, a pre-amplifier, and/or an amplifier; and one or more label probes, wherein each label probe comprises a detectable label. In some embodiments, the signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the kit further comprises a blocking agent, a crosslinking agent, a protease, or any combination thereof.

In some embodiments, the kit further comprises a conjugation reagent for conjugation of the oligonucleotide to the antibody. In some embodiments, the kit further comprises instructions for conjugating the oligonucleotide to the antibody. In some embodiments, the kit further comprises instructions for carrying out a method of detecting the target protein in the biological sample.

In some embodiments, the kit is for detecting two or more target proteins in the biological sample, and the kit comprises (i) a plurality oligonucleotides, wherein each oligonucleotide comprises a reactive moiety for conjugation to an antibody; and (ii) a plurality of signal-generating complexes, wherein each signal-generating complex comprises a nucleic acid component capable of hybridizing to one of the oligonucleotides.

In another aspect, provided herein is a kit for detecting a target nucleic acid and a target protein in a biological sample, comprising (i) an antibody or a fragment thereof, wherein the antibody or the fragment thereof is covalently attached to an oligonucleotide; (ii) a first signal-generating complex, wherein the signal-generating complex comprises a nucleic acid component capable of hybridizing to the oligonucleotide; (iii) one or more target probe(s) capable of hybridizing to the target nucleic acid; and (iv) a second signal-generating complex capable of hybridizing to the one or more target probe(s), wherein the second signal-generating complex comprises a nucleic acid component capable of hybridizing to the one or more target probe(s).

In some embodiments, the antibody or fragment thereof is selected from a monoclonal antibody, a multispecific antibody, a bispecific antibody, a trispecific antibody, a tetravalent antibody, a single-domain antibody, and a chimeric antibody. In some embodiments, the kit comprises a composition of polyclonal antibodies, wherein a plurality of antibodies in the composition are covalently attached to the oligonucleotide. In some embodiments, the antibody or fragment thereof is selected from a Fab, a scFv, a Fv, a scFv-Fc, a Fab′, a Fab′-SH, a F(ab′)₂, a diabody, a minibody, a tribody, a nanobody, and an affibody.

In some embodiments, the oligonucleotide has a length of about 10 to about 100 nucleotides.

In some embodiments, the oligonucleotide is covalently attached to the antibody via a linker. In some embodiments, the linker comprises an oligonucleotide sequence.

In some embodiments, the first signal-generating complex comprises: a pre-pre-amplifier, a pre-amplifier, and/or an amplifier; and one or more label probes, wherein each label probe comprises a detectable label.

In some embodiments, the first signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the antibody or the fragment thereof binds directly to the target protein.

In some embodiments, each of the one or more target probe(s) comprises a target (T) section and a label (L) section, wherein the T section is a nucleic acid sequence complementary to a section on the target nucleic acid and the L section is a nucleic acid sequence complementary to a section on the nucleic acid component of the second signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the second signal-generating complex.

In some embodiments, the second signal-generating complex comprises: a pre-pre-amplifier, a pre-amplifier, and/or an amplifier; and one or more label probes, wherein each label probe of the second signal-generating complex comprises a detectable label that is distinct from the detectable label of the first signal-generating complex.

In some embodiments, the second signal-generating complex comprises a pre-amplifier, an amplifier, and one or more label probes. In some embodiments, the detectable label comprises a fluorescent moiety or a chromogenic moiety. In some embodiments, the detectable label comprises a cleavable label.

In some embodiments, the kit further comprises a blocking agent, a crosslinking agent, a protease, or any combination thereof In some embodiments, the kit further comprises instructions for carrying out a method of detecting the target protein and the target nucleic acid in the biological sample.

In some embodiments, the kit further comprises the kit is for detecting two or more target proteins in the biological sample and/or two or more target nucleic acids in the biological sample.

Embodiments of the present disclosure also include a method for detecting a target protein in a biological sample, the method comprising (i) contacting the sample with at least one antibody or a fragment thereof, wherein the at least one antibody or the fragment thereof is covalently attached to an oligonucleotide; (ii) contacting the sample with a signal-generating complex, wherein the signal-generating complex comprises a nucleic acid component capable of hybridizing to the oligonucleotide; and (iii) detecting a signal from the signal-generating complex. In some embodiments, the method includes contacting the sample with a crosslinking agent after step (i) and before step (ii). In some embodiments, the crosslinking agent is glutaraldehyde.

In some embodiments, the at least one antibody or the fragment thereof comprises a primary antibody specific for an antigen on the target protein. In some embodiments, the at least one antibody or fragment thereof comprises at least two primary antibodies specific for different antigens on different target proteins. In some embodiments, the at least one antibody or fragment thereof comprises at least two primary antibodies specific for different antigens on the same target protein. In some embodiments, the at least one primary antibody or the at least two primary antibodies are covalently attached to the oligonucleotide.

In some embodiments, the at least one antibody or fragment thereof comprises at least one secondary antibody specific for an antigen on at least one primary antibody, wherein the at least one primary antibody is specific for an antigen on the target protein. In some embodiments, the at least one antibody or fragment thereof comprises at least two secondary antibodies specific for different antigens on at least two primary antibodies, wherein the at least two primary antibodies are specific for different antigens on different target proteins. In some embodiments, the at least one antibody or fragment thereof comprises at least two secondary antibodies specific for different antigens on at least two primary antibodies, wherein the at least two primary antibodies are specific for different antigens on the same target protein. In some embodiments, the at least one secondary antibody or the at least two secondary antibodies is/are covalently attached to the oligonucleotide.

In some embodiments, the method comprises (i) contacting the sample with a plurality of antibodies or fragments thereof, wherein the plurality of antibodies or fragments thereof comprises at least one primary antibody specific for a target protein and wherein the at least one primary antibody is covalently attached to an oligonucleotide; (ii) contacting the sample with a plurality of pre-amplifiers comprising binding sites for the oligonucleotide and a plurality of binding sites for an amplifier; (iii) contacting the sample with a plurality of amplifiers comprising binding sites for the pre-amplifiers and binding sites for label probes; (iv) contacting the sample with a plurality of label probes comprising a label and binding sites for the amplifiers, wherein the labels are cleavable; and (v) detecting the plurality of label probes, thereby detecting the target proteins.

In some embodiments, the method comprises (i) contacting the sample with a plurality of antibodies or fragments thereof, wherein the plurality of antibodies or fragments thereof comprises at least one primary antibody specific for a target protein and at least one secondary antibody specific for the primary antibody, and wherein the at least one secondary antibody is covalently attached to an oligonucleotide; (ii) contacting the sample with a plurality of pre-amplifiers comprising binding sites for the oligonucleotide and a plurality of binding sites for an amplifier; (iii) contacting the sample with a plurality of amplifiers comprising binding sites for the pre-amplifiers and binding sites for label probes; (iv) contacting the sample with a plurality of label probes comprising a label and binding sites for the amplifiers, wherein the labels are cleavable; and (v) detecting the plurality of label probes, thereby detecting the target protein.

In some embodiments, the method further comprises cleaving the labels from the plurality of label probes; (vi) contacting the sample with at least a second plurality of label probes comprising a label and binding sites for the amplifiers; and (xi) detecting the second plurality of label probes, thereby detecting a second target protein.

Embodiments of the present disclosure also include a kit for detecting a target protein in a biological sample, comprising (i) at least one antibody or a fragment thereof, wherein the at least one antibody or the fragment thereof is covalently attached to an oligonucleotide; and (ii) a signal-generating complex, wherein the signal-generating complex comprises a nucleic acid component capable of hybridizing to the oligonucleotide.

In some embodiments, the at least one antibody or the fragment thereof comprises a primary antibody specific for an antigen on the target protein. In some embodiments, the at least one antibody or fragment thereof comprises at least two primary antibodies specific for different antigens on different target proteins. In some embodiments, the at least one antibody or fragment thereof comprises at least two primary antibodies specific for different antigens on the same target protein. In some embodiments, the at least one primary antibody or the at least two primary antibodies are covalently attached to the oligonucleotide.

In some embodiments, the at least one antibody or fragment thereof comprises at least one secondary antibody specific for an antigen on at least one primary antibody, wherein the at least one primary antibody is specific for an antigen on the target protein. In some embodiments, the at least one antibody or fragment thereof comprises at least two secondary antibodies specific for different antigens on at least two primary antibodies, wherein the at least two primary antibodies are specific for different antigens on different target proteins. In some embodiments, the at least one antibody or fragment thereof comprises at least two secondary antibodies specific for different antigens on at least two primary antibodies, wherein the at least two primary antibodies are specific for different antigens on the same target protein. In some embodiments, the at least one secondary antibody or the at least two secondary antibodies is/are covalently attached to the oligonucleotide.

### BRIEF DESCRIPTION OF THE DRAWINGS

FIGS. 1A-1B: FIG. 1A includes schematic illustrations of the methods of the present disclosure involving detecting one or more target proteins (e.g., ProteinScope) in a sample (right panel), as compared to traditional immunohistochemistry (left panel). FIG. 1B includes a schematic illustration of a representative workflow for the methods of the present disclosure (e.g., ProteinScope), which can include several optional steps (e.g., crosslink, protease treatment, and/or blocking steps).

FIGS. 2A-2B: FIG. 2A includes a schematic illustration of the methods of the present disclosure involving the detection of one or more target proteins (e.g., ProteinScope) and one or more target nucleic acids (e.g., RNAscope®) in a sample (e.g., duplex and multiplex formats). FIG. 1B includes a representative schematic illustration of a corresponding workflow.

FIG. 3 includes a schematic illustration of a representative sulfo-SMCC crosslinking workflow.

FIG. 4: Representative image demonstrating effective Ki67 target protein detection in cancer samples using the methods of the present disclosure (e.g., ProteinScope), as compared to conventional IHC.

FIGS. 5A-5C: Representative images demonstrating target protein detection using the methods of the present disclosure (e.g., ProteinScope) on cancer samples using sulfo-SMCC conjugated antibodies against Ki67 (FIG. 5A), CD8 (FIG. 5B), and INSR (FIG. 5C). “IHC” indicates use of conventional immunohistochemistry methods, while “PS” indicates use of ProteinScope methods. “Higher labeling,” “lower labeling,” and “single labeling” refer to different degrees of labeling (oligo/antibody ratio).

FIGS. 6A-6B: Representative image demonstrating target protein detection using the methods of the present disclosure (e.g., ProteinScope) on cancer samples using sulfo-SMCC conjugated antibodies against Ki67 (FIG. 6A) and INSR (FIG. 6B), with various conjugate crosslinker lengths (e.g., PEG4, PEG8, and PEG12).

FIGS. 7A-7B: Representative image demonstrating Ki67 target protein detection in cancer samples using the methods of the present disclosure (e.g., ProteinScope), including a blocking step (e.g., tRNA and salmon sperm DNA in FIG. 7A; BSA in FIG. 7B) before application of the Ki67 target antibody.

FIGS. 8A-8C: FIG. 8A includes a schematic illustration of the various types of antibodies that can be used to detect a target antigen using the methods of the present disclosure, including but not limited to, monoclonal antibodies, multispecific antibodies, bispecific antibodies, tri specific antibodies, tetravalent antibodies, single-domain antibodies, and chimeric antibodies. FIG. 8B includes a schematic illustration of an oligo-conjugated target antibody (e.g., monoclonal antibody, single-domain antibody) bound to a signal generating complex. FIG. 8C includes a schematic illustration of the use of multiple target antibodies (e.g., multiplexing).

FIG. 9: Representative schematic illustration of the signal generating complexes used in the methods of the present disclosure, which allow for scaled amplification reactions based on the number (or tiers) of oligos used to generate the complexes.

FIGS. 10A-10D: FIG. 10A includes representative images demonstrating detection of target protein (Ki76) and target RNA (Hs-PPIB and Hs-UBC) using the methods of the present disclosure (e.g., ProteinScope and RNAscope®) on a single sample (multiplexing). FIG. 10B includes representative images demonstrating multiplex staining using the ProteinScope workflow showing one protein marker (PD1) in T5 channel (green) and three mRNA markers in T6 (red), T7 (white) and T8 (pink) channels on FFPE human tonsil tissue. The mRNA markers are shown in italics. FIG. 10C includes representative images demonstrating multiplex staining using the ProteinScope workflow showing six protein markers (CD68, PD1, CD4, FOXP3, CD8, CD3e) and six mRNA markers (shown in italics) on FFPE human tonsil tissue. Bottom panel shows overlay of protein markers only. FIG. 10D includes representative images demonstrating multiplex staining using the ProteinScope workflow showing eleven protein markers (CD8a, SDHB, Keratin-14, GAPDH, CD68, CD3e, PD1, FOXP3, Vimentin, EpCAM, CD4) and one mRNA marker (Hs-POLR2A, shown in italics) on FFPE human head and neck cancer tissue. Bottom right panel shows overlay of protein markers only.

FIGS. 11A-11B: Representative workflow diagrams of the image processing methods to reduce background signals, described with reference to the images being processed (FIG. 11A) and the general steps of the method (FIG. 11B), according to one embodiment of the present disclosure.

FIGS. 12A-12F: FIG. 12A includes a representative schematic of the concept of signal detection using oligo-conjugated secondary antibodies in which amplification of oligos through signal-generating complexes enables host-independent protein detection. FIGS. 12B and 12C include representative workflows for dual detection of two primary antibodies from a rabbit host. FIG. 12D includes representative images demonstrating similar staining between ProteinScope utilizing oligo-conjugated antibodies and traditional immunohistochemistry utilizing secondary antibody-based detection. FIG. 12E includes representative images demonstrating dual protein staining (right panel) with anti-rabbit secondary antibody conjugated to T1 and T3 channel oligos. CD3e and PD1 primary antibodies from rabbit host are mixed with oligo-conjugated anti-rabbit secondary antibodies in individual tubes, quenched with rabbit serum, and then functionally tested using the ProteinScope workflow either in singleplex staining (left and middle panel) or as a cocktail (right panel). FIG. 12F includes representative images demonstrating triple protein staining for three rabbit anti-human antibodies, GFAP, NeuN and IBA1, detected using oligo-conjugated secondary antibodies in the ProteinScope workflow. Corresponding immunofluorescence controls are shown in individual panels.

FIGS. 13A-13D: FIG. 13A includes representative images demonstrating that PD1 protein signal detection (channel T5) is stronger with glutaraldehyde cross-linking after the primary antibody step. Glutaraldehyde cross-linking also picks up more PD1 positive cells than 10% NBF. FIG. 13B includes representative images demonstrating that protein signal detection for CD68 marker was stronger than control (10% NBF) at all concentrations of glutaraldehyde tested (GA = glutaraldehyde). FIG. 13C includes representative images demonstrating shorter cross-linking time with glutaraldehyde retains increased sensitivity compared to control (10% NBF). FIG. 13D includes representative images demonstrating that dilution of glutaraldehyde in 10% NBF reduces autofluorescence.

### DETAILED DESCRIPTION

The present disclosure is directed to methods of detecting a protein in a biological sample, and methods of detecting a protein and a nucleic acid in a biological sample, and kits for conducting such methods. Rather than using traditional IHC methods, the methods disclosed herein use an antibody or a fragment thereof that is conjugated to an oligonucleotide. A signal-generating complex includes a nucleic acid component that can hybridize to the oligonucleotide, to provide a detectable signal that indicates the presence of the target protein in the sample. In some embodiments, the signal-generating complexes used in the methods disclosed herein provide highly amplified signals that enhance detection of the target proteins. In some embodiments, methods disclosed herein for detecting a protein in a biological sample are referred to as “ProteinScope.”

a. Definitions

As used herein, the term “in situ hybridization” or “ISH” refers to a technique for localizing and visualizing specific target nucleic acids with the preservation of morphology of the source samples.

As used herein, the term “immunohistochemistry” or “IHC” generally refers to a technique for detecting proteins of interest in source samples utilizing antibodies, with the preservation of morphology of the source samples (e.g., tissue samples). As used herein, the term “immunocytochemistry” or “ICC” generally refers to a technique for detecting proteins of interest in source samples utilizing antibodies, with the preservation of morphology of the source samples (e.g., isolated or cultured intact cells, including tissue culture cell lines, either adherent or in suspension). Immunofluorescence (IF) refers to fluorescent labeling, thus it is also encompassed in the terms IHC and ICC. ICC, IHC, and IF assays can be used in conjunction with the imaging processing methods of the present disclosure, as described further herein, including facilitating quantitative and/or qualitative assessments of a target-of-interest in a sample. ICC, IHC, and IF assays can also be performed in conjunction with an in situ hybridization as part of an integrated co-detection process to detect targets-of-interest, which can also include performing the imaging processing methods of the present disclosure.

As used herein, the term “crosslink” refers to a process of binding two or more molecules together. The “crosslinking agent” or equivalent refers to agents containing two or more chemically reactive ends that attach themselves to the functional groups found in proteins and other molecules. Specifically, if the crosslinking agent is formaldehyde or its equivalent, a nucleophilic group on an amino acid or nucleic acid base forms a covalent bond with formaldehyde, which is stabilized in a second step that involves another functional group, often on another molecule, leading to formation of a methylene bridge. If the crosslinking agent is an oxidizing agent, it can react with the side chains of proteins and other biomolecules, allowing the formation of crosslinks that stabilize tissue structure.

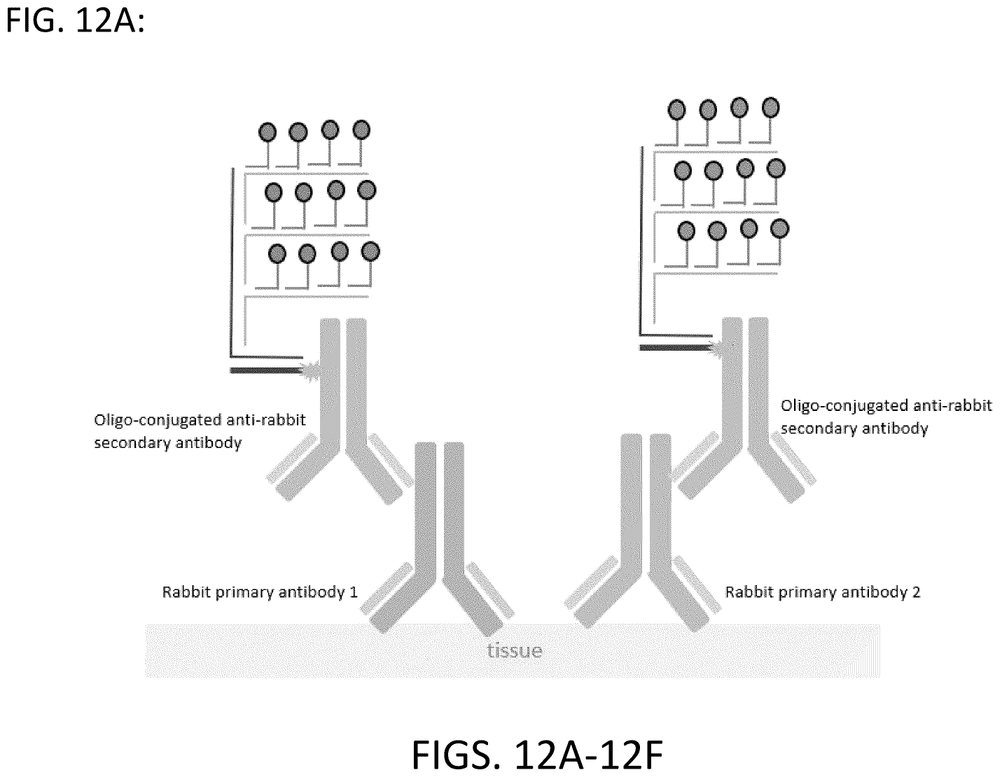

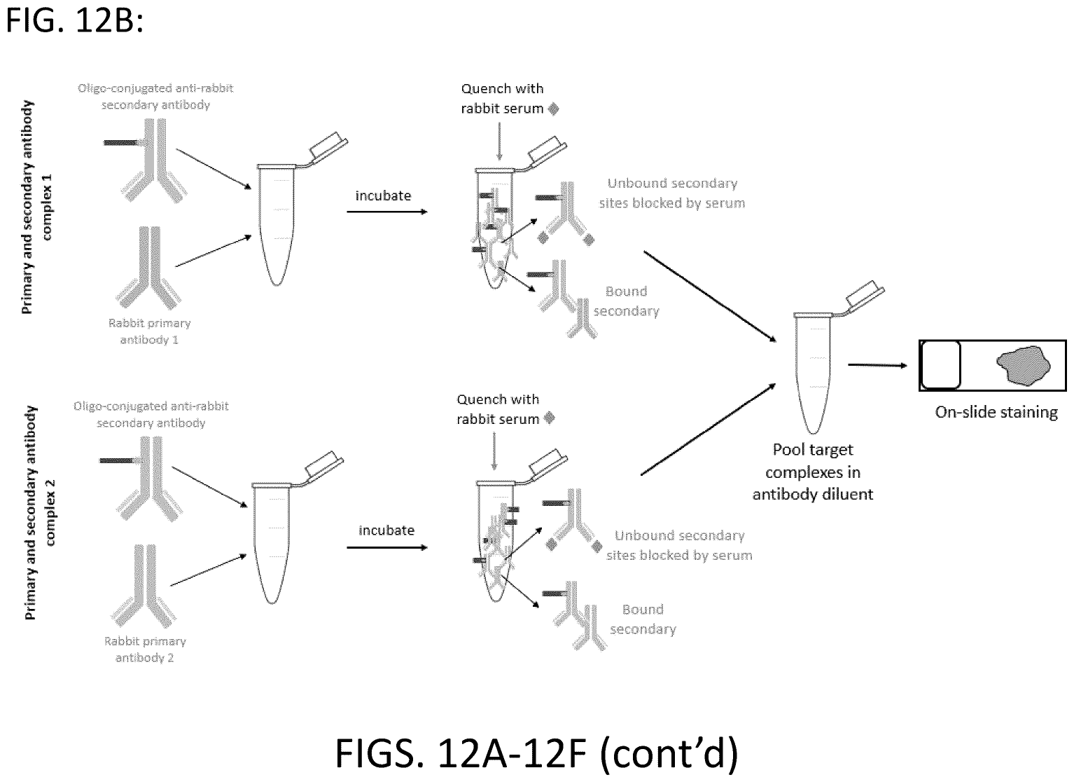

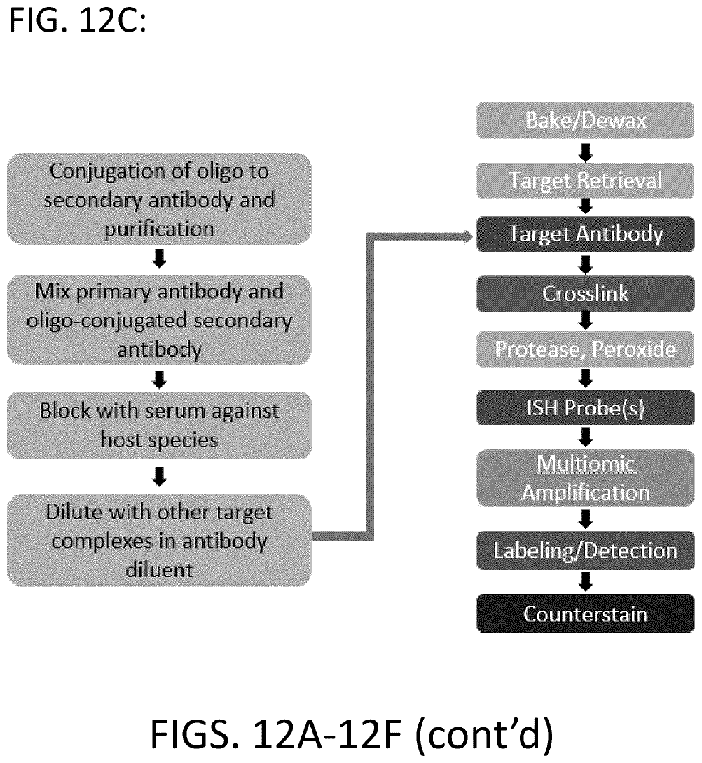

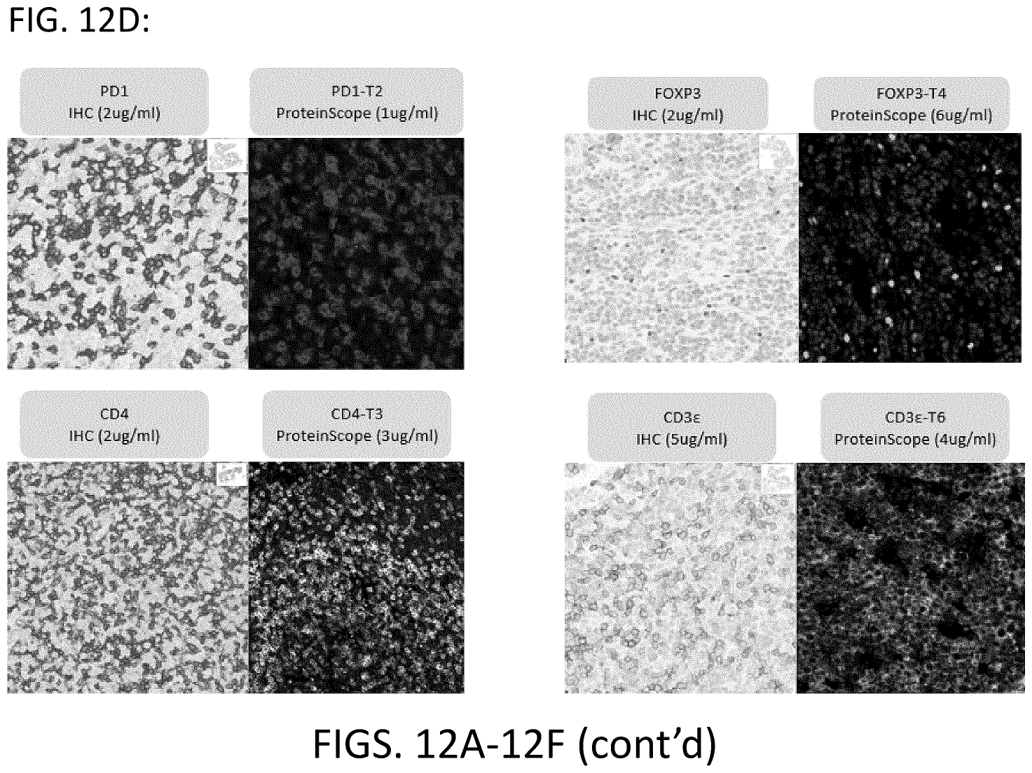

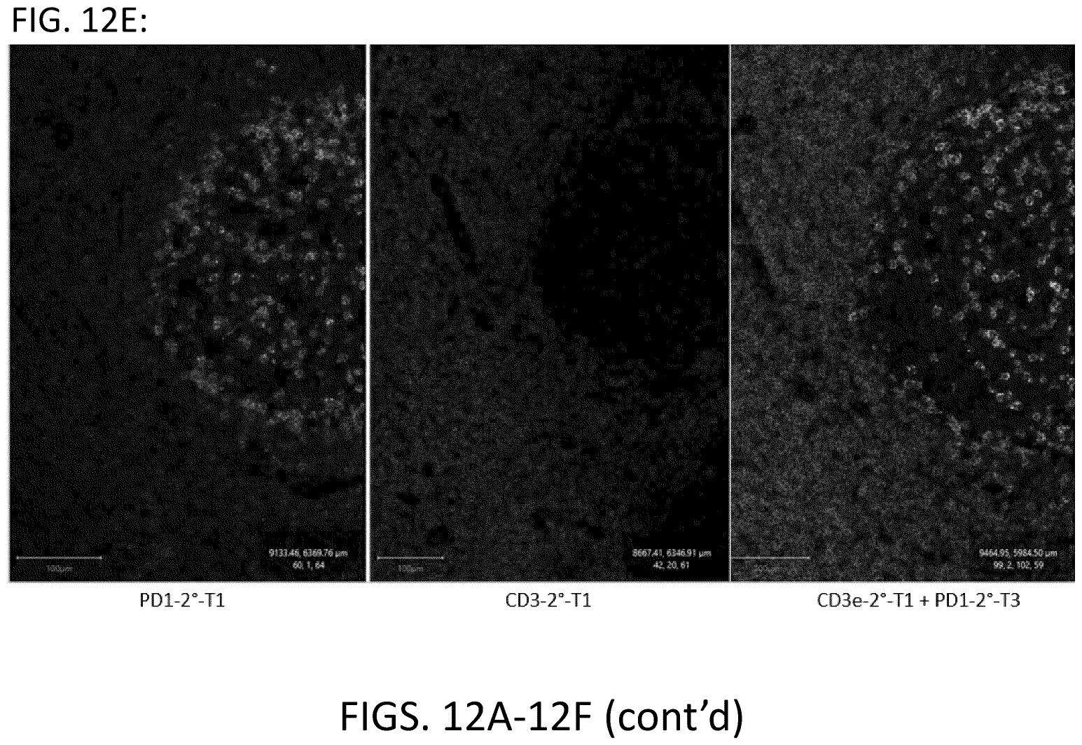

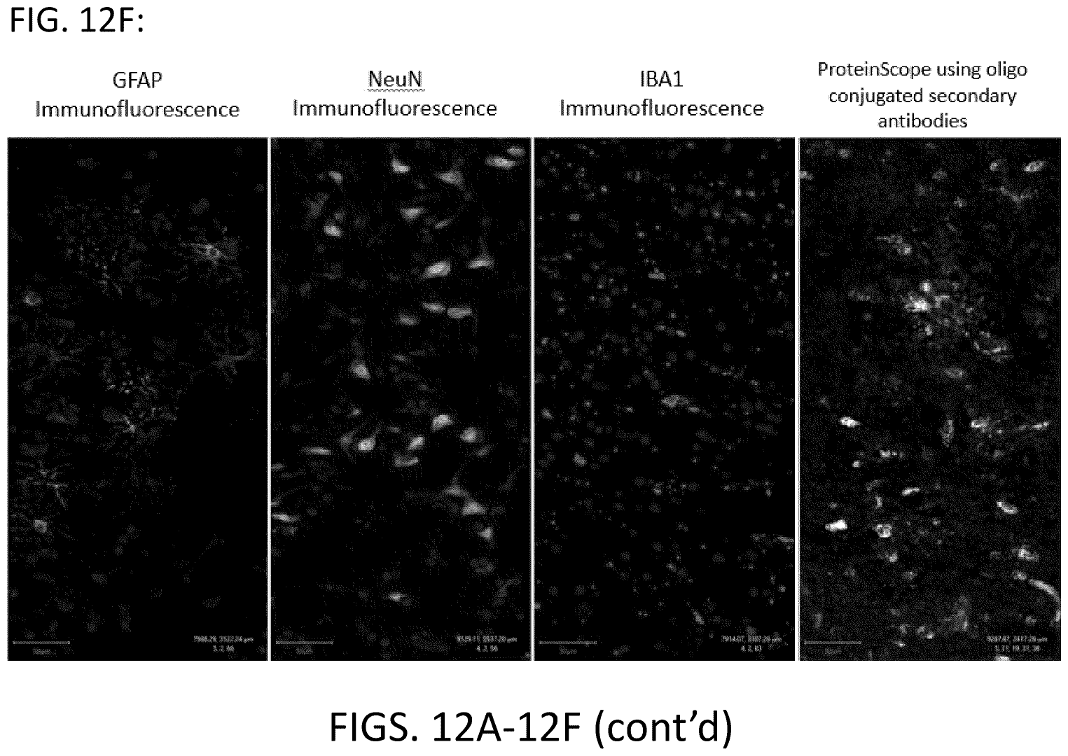

As used herein, the term “primary antibody” refers to an antibody that binds directly to the antigen of interest. As used herein, the term “secondary antibody” refers to an antibody that is conjugated to a moiety that can be used for detection, such as a detectable label, or a moiety to which a detectable label can bind (see, e.g., FIGS. 12A-12F). In some embodiments, a secondary antibody is conjugated to an oligonucleotide that is capable of hybridizing to a nucleic acid component of a signal-generating complex. In some embodiments, the secondary antibody provided herein binds directly to the primary antibody. In other embodiments, the secondary antibody provided herein binds indirectly to the primary antibody, e.g., by binding to another antibody that recognizes the primary antibody.

As used herein, the term “fixation” or “fixing” when made in reference to fixing a biological sample in the ISH process refers to a procedure to preserve a biological sample from decay due to, e.g., autolysis or putrefaction. It terminates any ongoing biochemical reactions and may also increase the treated tissues' mechanical strength or stability.

As used herein, the term “one or more” refers to, for example, 1 or more, 2 or more, 3 or more, 4 or more, 5 or more, 6 or more, 7 or more, 8 or more, 9 or more, 10 or more, 20 or more, 30 or more, 40 or more, 50 or more, or a greater number, if desired for a particular use.

The terms “detecting” as used herein generally refer to any form of measurement, and include determining whether an element is present or not. This term includes quantitative and/or qualitative determinations.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, cells, and exosomes isolated from a mammal. Exemplary biological samples include but are not limited to cell lysate, a cell, a cell culture, a cell line, a tissue, oral tissue, gastrointestinal tissue, an organ, an organoid, a biological fluid, a blood sample, a urine sample, a skin sample, and the like. Preferred biological samples include, but are not limited to, whole blood, partially purified blood, PBMC, tissue biopsies, and the like.

The term “probe” as used herein refers to a capture agent that is directed to a specific target mRNA sequence. Accordingly, each probe of a probe set has a respective target mRNA sequence. In some embodiments, a probe can be used individually. In other embodiments, a probe can be used among a probe set. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

b. Methods of Detecting a Target Protein

In one aspect, provided herein is a method for detecting a target protein in a biological sample, comprising: (i) contacting the sample with an antibody or a fragment thereof, wherein the antibody or the fragment thereof is covalently attached to an oligonucleotide; (ii) contacting the sample with a signal-generating complex, wherein the signal-generating complex comprises a nucleic acid component capable of hybridizing to the oligonucleotide; and (iii) detecting a signal from the signal-generating complex.

Step (i) involves use of an antibody or a fragment thereof, which is covalently attached to an oligonucleotide. The antibody or the fragment thereof binds to the target protein in the sample, and the oligonucleotide provides a binding site for a signal-generating complex, as will be further discussed below. Any suitable antibody can be used. In some embodiments, the antibody or fragment thereof is selected from a monoclonal antibody, a multispecific antibody, a bispecific antibody, a trispecific antibody, a tetravalent antibody, a single-domain antibody, and a chimeric antibody. In some embodiments, the antibody or fragment thereof is selected from a Fab, a scFv, a Fv, a scFv-Fc, a Fab′, a Fab′-SH, a F(ab′)₂, a diabody, a minibody, a tribody, a nanobody, and an affibody. In some embodiments, step (i) involves use of a composition of polyclonal antibodies, in which a plurality of antibodies in the composition are conjugated to the oligonucleotide.

The oligonucleotide that is covalently attached to the antibody or fragment thereof can have a length of about 10 to about 100 nucleotides. In some embodiments, the oligonucleotide has a length of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In some embodiments, the oligonucleotide has a length of about 10 to about 50 nucleotides. In some embodiments, the oligonucleotide has a length of about 12 to about 16 nucleotides (e.g., 14 nucleotides). In some embodiments, the oligonucleotide has a length of about 26 to about 30 oligonucleotides (e.g., 28 nucleotides). In some embodiments, the oligonucleotide has a length of about 40 to about 60 nucleotides (e.g., 50 nucleotides).

As will be further discussed below, the oligonucleotide sequence is selected such that a nucleic acid component of the signal-generating complex is capable of hybridizing to the oligonucleotide. In some embodiments, the oligonucleotide has a sequence that is complementary to a sequence of a nucleic acid component of the signal-generating complex. For example, in some embodiments, the oligonucleotide has a sequence that is complementary to a sequence of a nucleic acid component of the signal-generating complex over a sequence of about 10 to about 100 nucleotides, e.g., a sequence of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In some embodiments, the oligonucleotide has a sequence that is complementary to a sequence of a nucleic acid component of the signal-generating complex over a sequence of about 10 to about nucleotides, about 12 to about 16 nucleotides (e.g., 14 nucleotides, about 26 to about 30 oligonucleotides (e.g., 28 nucleotides), or about 40 to about 60 nucleotides (e.g., 50 nucleotides).

The oligonucleotide is covalently attached to the antibody or the fragment thereof. In some embodiments, the covalent attachment is via a direct bond between the antibody or the fragment thereof and the oligonucleotide. In some embodiments, the oligonucleotide is covalently attached to the antibody via a linker.

General methods of conjugating oligonucleotides to antibodies are known to those skilled in the art. For example, a typical conjugation method includes use of a linker compound that includes two distinct reactive moieties, which react with different types of functional groups (e.g., one group that reacts with an amine, such as an activated ester group, and one group that reacts with a thiol, such as a maleimide group). Such reactive moieties used in conjugation reactions are well-known to those skilled in the art, and include activated esters such as succinimidyl and sulfosuccinimidyl esters and pentafluorophenyl esters, maleimides, azides, alkynes, hydrazines, isocyanates, isothiocyanates, haloacetamides, and the like. Methods of installing such reactive groups are well-known to those skilled in the art. As one non-limiting example, an amino group can be installed at the 5′-end of an oligonucleotide via phosphoramidite chemistry.

In some embodiments, the antibody is first reacted with the linker compound to provide a functionalized antibody, which is subsequently reacted with an oligonucleotide to provide the oligonucleotide-labeled antibody. In other embodiments, the oligonucleotide is first reacted with the linker compound to provide a functionalized oligonucleotide, which is subsequently reacted with an antibody to provide the oligonucleotide-labeled antibody.

Some oligonucleotide-antibody conjugation reagents or linkers are commercially available, and some are sold as parts of commercial kits. Exemplary commercially available linker compounds include those shown in Scheme 1, such as sulfosuccinimidyl-4-(N-maleimidomethyl) cyclohexane-1-carboxylate (sulfo-SMCC), succinimidyl-4-(N-maleimidomethyl) cyclohexane-1-carboxylate (SMCC), PEGylated crosslinkers such as SM(PEG)ₙ compounds (succinimidyl-([N-maleimidopropionamido](CH₂CH₂O)ₙ) esters), and 6-hydrazinonicotinate (HyNic) containing linkers such as S-HyNic.

In some embodiments, the commercial linkers can be used directly to conjugate the antibody to the oligonucleotide. In other embodiments, the antibody and/or the oligonucleotide must be first derivatized with a specific functional group prior to reaction with the linker compound. For example, the antibody can be reacted with a 2-iminothiolane to install a thiol group for reaction with a maleimide group. As another example, the oligonucleotide or antibody can be reacted with succinimidyl-4-formylbenzamide to install an aldehyde group for reaction with a HyNic-containing linker compound.

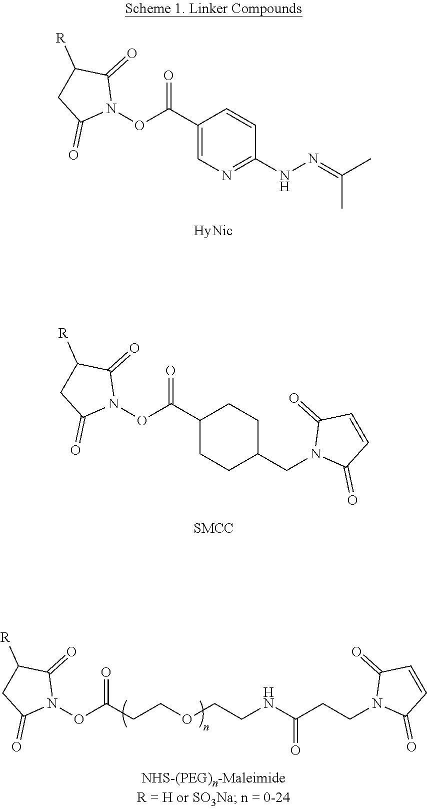

Other known linker chemistries involve separate functionalizations of the antibody and the oligonucleotide followed by a reaction to generate the linker moiety. Examples include installation of an azide-containing moiety on one compound and an alkyne-containing moiety on the other, for linkage via click chemistry (e.g., either copper-catalyzed or copper-free click chemistry).

Accordingly, in some embodiments, the linker comprises a moiety selected from:

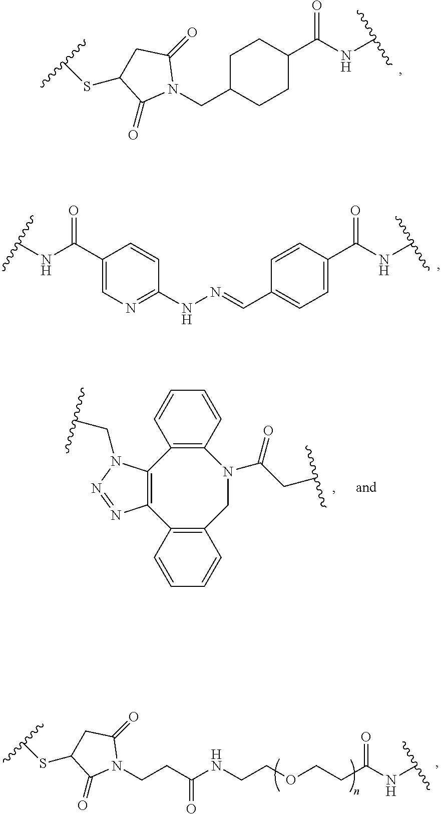

wherein n is 0, 1, 2, 3, 4, 5, 6, 7, 8, 9, 10, 11, 12, 13, 14, 15, 16, 17, 18, 19, 20, 21, 22, 23, or 24.

In some embodiments, the linker can include one or more nucleotides; e.g., the linker can comprise an oligonucleotide sequence. Such a sequence may be considered separate from the oligonucleotide sequence to which the nucleic acid component of the signal-generating complex can hybridize. For example, in some embodiments, the linker can include one or more thymine groups. In some embodiments, the linker is a 5T linker.

The linker can include additional atoms or groups; for example, if the antibody is reacted with 2-iminothiolane, it is understood that the linker will further include atoms derived from such reaction. Accordingly, in some embodiments, the linker further comprises one or more additional groups selected from —CH₂—, —O—, —NH—, —S—, —C(═O)—, —C(═NH)—, and any combination thereof (e.g., combinations of such moieties could include ester groups (—C(═O)O—), amide groups (—C(═O)NH—), carbamate groups (—NHC(═O)O—), ethylene glycol groups (—CH₂CH₂O—), and the like.

In some embodiments, the linker comprises an antibody-binding domain. In some embodiments, an antibody binding domain (AbBD) comprises Protein A, Protein G, Protein L, CD4, or a fragment thereof. In some embodiments, the antibody-binding domain is an engineered antibody-binding domain, such as to include a non-natural amino acid, a photoreactive group, or a crosslinker. In some embodiments, the antibody binding domain is operably linked to a photoreactive amino acid group, for example, benzoylphenylalanine (BPA), resulting in a photoreactive antibody binding domain (pAbBD). In some embodiments, the antibody-binding domain (AbBD) is operably linked to a photoreactive amino acid which is operably linked to an antibody or a fragment thereof. See, for example, U.S. Pat. Nos. 11,156,608 and 11,123,440.

The method is not limited by the order in which steps (i) and (ii) are performed. In some embodiments, step (i) and step (ii) are performed simultaneously. In some embodiments, step (i) is performed before step (ii). In some embodiments, step (ii) is performed before step (i).

In some embodiments, step (i) comprises contacting the sample with the antibody or the fragment thereof for about 10 minutes to about 48 hours, or about 15 minutes to about 120 minutes. For example, in some embodiments, step (i) comprises contacting the sample with the antibody or the fragment thereof for about 10 minutes, about 15 minutes, about 20 minutes, about 25 minutes, about 30 minutes, about 35 minutes, about 40 minutes, about 45 minutes, about 50 minutes, about minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours.

In some embodiments, step (i) comprises contacting the sample with the antibody or the fragment thereof at a temperature of about 4° C. to about 75° C., or about 4° C. to about 25° C. For example, in some embodiments, step (i) comprises contacting the sample with the antibody or the fragment thereof at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, step (i) comprises contacting the sample with the antibody or the fragment thereof at room temperature.

In some embodiments, the antibody or the fragment thereof binds directly to the target protein in the biological sample. In such embodiments, the method does not require use of a primary antibody that binds directly to the target protein. In other embodiments, the antibody or the fragment thereof binds indirectly to the target protein in the biological sample. In such embodiments, the method can further comprise a step of contacting the sample with a primary antibody prior to step (i), wherein the primary antibody binds directly to the target protein; the antibody or the fragment thereof that is covalently attached to the oligonucleotide then binds to the primary antibody.

In some embodiments, the method further comprises contacting the sample with a blocking agent prior to step (i), to minimize non-specific binding that can result in unwanted background signals. Suitable blocking agents include those that comprise DNA, RNA, or protein. For example, in some embodiments, the blocking agent comprises DNA, such as salmon sperm DNA, herring sperm DNA, or calf thymus DNA. In some embodiments, the blocking agent comprises RNA, such as tRNA. In some embodiments, the blocking agent comprises a protein; for example, in some embodiments, the blocking agent comprises bovine serum albumin (BSA), casein, an animal serum such as normal goat serum, normal swine serum, normal chicken serum, or a fish serum such as steelhead salmon serum. In some embodiments, the blocking agent is a non-animal protein blocking agent, such as one comprising a plant protein. Non-animal protein blocking agents are commercially available, e.g., from G-Biosciences® (NAP-BLOCKER™) and Vector Laboratories (Animal-Free Blocker®).

In some embodiments, the method further comprises contacting the sample with a crosslinking agent after step (i) and before step (ii). Such a step has been found to preserve and even improve signals in IHC assays when conducted after incubation with a primary antibody and before incubation with a secondary antibody, such as when samples are exposed to protease treatment (see WO 2021/226311). In some embodiments, the method comprises contacting the sample with a crosslinking agent after step (i) and before step (ii), where step (i) includes contacting the sample with a complex comprising primary antibodies from the same or multiple hosts that are first individually complexed with oligo-conjugated respective secondary antibodies (see, e.g., FIGS. 12A-12F). In certain embodiments, the crosslinking agent is a fixative. In some embodiments, the crosslinking agent is selected from neutral-buffered formalin (NBF), formaldehyde, glutaraldehyde, glyoxal, acrolein, osmium tetroxide, a permanganate fixative (e.g., potassium permanganate), a dichromate fixative (e.g., potassium dichromate), chromic acid, and a mixture of any thereof. In particular embodiments, the crosslinking agent is NBF, such as about 1% to about 20% NBF (e.g., 10% NBF). In some embodiment, the crosslinking agent is a mixture of any of the above fixatives, with or without additional compounds. For example, in some embodiments, the crosslinking agent is selected from: Bouin's fixative (picric acid, formaldehyde, and acetic acid), a mixture of formaldehyde and glutaraldehyde; FAA (ethanol, acetic acid, and formaldehyde); periodate-lysine-paraformaldehyde (PLP) (paraformaldehyde, L-lysine, and INaO₄); phosphate buffered formalin (PBF); formal calcium (formaldehyde and calcium chloride); formal saline (formaldehyde and sodium chloride); zinc formalin (formaldehyde and zinc sulfate); Helly's fixative (formaldehyde, potassium dichromate, sodium sulphate, and mercuric chloride); Hollande's fixative (formaldehyde, copper acetate, picric acid, and acetic acid); Gendre's solution (formaldehyde, ethanol, picric acid, and glacial acetic acid); alcoholic formalin (formaldehyde, ethanol, and calcium acetate); and formol acetic alcohol (formaldehyde, glacial acetic acid, and ethanol). In some embodiments, the crosslinking agent comprises a polymer with at least two reactive functional groups, such as succinimidyl esters. In some embodiments, the crosslinking agent is a bis(succinimidyl) polyethylene glycol. In some embodiments, the crosslinking agent is provided as an aqueous solution at a pH of about 6 to about 9, e.g., about 6.0, about 6.5, about 7.0, about 7.5, about 8.0, about 8.5, or about 9.0. In embodiments in which two or more crosslinking agents are used, the sample may be contacted with the two or more crosslinking agents either simultaneously or consecutively.

In some embodiments, the crosslinking agent is glutaraldehyde, or a derivative thereof. In some embodiments, the method comprises contacting the sample with glutaraldehyde, or a derivative thereof, after step (i) and before step (ii). In some embodiments, the method comprises using glutaraldehyde at a concentration that ranges from about 0.001% to about 5%. In some embodiments, the method comprises using glutaraldehyde at a concentration that ranges from about 0.001% to about 2.5%. In some embodiments, the method comprises using glutaraldehyde at a concentration that ranges from about 0.001% to about 1%. In some embodiments, the method comprises using glutaraldehyde at a concentration that ranges from about 0.001% to about 0.5%. In some embodiments, the method comprises using glutaraldehyde at a concentration that ranges from about 0.001% to about 0.01%. In some embodiments, the method comprises using glutaraldehyde at a concentration that ranges from about 0.01% to about 5%. In some embodiments, the method comprises using glutaraldehyde at a concentration that ranges from about 0.1% to about 5%. In some embodiments, the method comprises using glutaraldehyde at a concentration that ranges from about 0.5% to about 5%. In some embodiments, the method comprises using glutaraldehyde at a concentration that ranges from about 1.0% to about 5%. In some embodiments, the method comprises using glutaraldehyde at a concentration that ranges from about 2.5% to about 5%. In some embodiments, the method comprises using glutaraldehyde at any of these concentrations in 10% neutral buffered formalin (NBF) or any similar fixative solution. In some embodiments, the method comprises using glutaraldehyde at any of these concentrations in 15% NBF, 14% NBF, 13% NBF, 12% NBF, 11% NBF, 10% NBF, 9% NBF, 8% NBF, 7% NBF, 6% NBF, 5% NBF, 4% NBF, 3% NBF, 152 NBF, or 1% NBF.

In some embodiments, the step of contacting the sample with a crosslinking agent is conducted at a temperature of about 0° C. to about 100° C., about 1° C. to about 90° C., about 2° C. to about 80° C., about 3° C. to about 70° C., or about 4° C. to about 60° C., e.g., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., about 55° C., about 60° C., about 65° C., about 70° C., about 75° C., about 80° C., about 85° C., about 90° C., about 95° C., about or 100° C. In some embodiments, the step of contacting the sample with a crosslinking agent is conducted for about 5 minutes to about 48 hours, about 5 minutes to about 24 hours, or about 15 minutes to about 18 hours, e.g., about 5 minutes, about 10 minutes, about 15 minutes, about 20 minutes, about 25 minutes, about 30 minutes, about minutes, about 40 minutes, about 45 minutes, about 50 minutes, about 55 minutes, about 60 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours.

In some embodiments, the method further comprises treating the biological sample with a protease after treating the biological sample with the crosslinking agent and before step (ii). This step can be used to digest certain proteins that surround the target. In some embodiments, the protease is selected from trypsin, proteinase K, pepsin, pronase, endoproteinase AspN, and endoproteinase GluC. In some embodiments, the method further comprises treating the biological sample with hydrogen peroxide after treating the biological sample with the crosslinking agent and before step (ii). This step is particularly useful when horseradish peroxidase (HRP) will be used as detection enzyme in the later steps, as the hydrogen peroxide inactivates endogenous HRP activity in the sample, thus reducing assay background. In other embodiments, for example, a tissue clearing step can be performed (e.g., lipid removal and protein denaturation) with 2-8% SSC in boric acid buffer before application of an antibody. This step can be performed to replace protease treatment, e.g., for RNA detection, since protease may have negative impact on antigen stability.

Step (ii) of the method comprises contacting the sample with a signal-generating complex (SGC), wherein the signal-generating complex comprises a nucleic acid component capable of hybridizing to the oligonucleotide. In some embodiments, the SGC is the same or similar SGC used in RNAscope®, which is described in more detail in, e.g., U.S. Pat. Nos. 7,709,198, 8,604,182, and 8,951,726. Specifically, RNAscope® uses specially designed oligonucleotide probes in combination with a branched-DNA-like SGC to reliably detect RNA under standard bright-field microscopy (Anderson et al., J. Cell. Biochem. 117(10):2201-2208 (2016); Wang et al., J. Mol. Diagn. 14(1):22-29 (2012)). For example, in some embodiments, the SGC includes a pre-pre-amplifier, a pre-amplifier, and/or an amplifier, and one or more label probes, wherein each label probe comprises a detectable label. In some embodiments, the SGC comprises a pre-amplifier, an amplifier, and one or more label probes, wherein each label probe comprises a detectable label. When used in methods of detecting a protein described herein, rather than binding to one or more target probe(s) that bind to a target nucleic acid as in RNAscope®, the SGC instead binds to the oligonucleotide that is conjugated to the antibody or the fragment thereof.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target. In general, the label probe can hybridize to a nucleic acid that is in turn hybridized to the target, or to one or more other nucleic acids that are hybridized to the target. Thus, the label probe can comprise a polynucleotide sequence that is complementary to a polynucleotide sequence, particularly a portion, of the target. Alternatively, the label probe can comprise at least one polynucleotide sequence that is complementary to a polynucleotide sequence in an amplifier, pre-amplifier, or pre-pre-amplifier in an SGC.

In some embodiments, the SGC provided herein comprises additional components such an amplifier, a pre-amplifier, and/or a pre-pre-amplifier.

As used herein, an “amplifier” is a molecule, typically a polynucleotide, that is capable of hybridizing to multiple label probes. Typically, the amplifier hybridizes to multiple identical label probes. The amplifier can also hybridize directly to the target, or to another nucleic acid bound to the target such as a pre-amplifier. For example, the amplifier can hybridize to the target and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198, and U.S. Publication Nos. 2008/0038725 and 2009/0081688, each of which is incorporated herein by reference.

As used herein, a “pre-amplifier” is a molecule, typically a polynucleotide, that serves as an intermediate binding component between the target and one or more amplifiers. Typically, the pre-amplifier hybridizes simultaneously to the target and to a plurality of amplifiers.

Exemplary pre-amplifiers are described, for example, in U.S. Pat. Nos. 5,635,352, 5,681,697, and 7,709,198, and U.S. Publication Nos. 2008/0038725, 2009/0081688 and 2017/0101672, each of which is incorporated by reference.

As used herein, a “pre-pre-amplifier” is a molecule, typically a polynucleotide, that serves as an intermediate binding component between the target and one or more pre-amplifiers. Typically, the pre-pre-amplifier hybridizes simultaneously to the target and to a plurality of pre-amplifiers. Exemplary pre-pre-amplifiers are described, for example, in U.S. Publication No. 2017/0101672, which is incorporated by reference.

As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In particular embodiments, the label comprises a fluorescent moiety or a chromogenic moiety. In a particular embodiment, the label is an enzyme. Exemplary enzyme labels include, but are not limited to horseradish peroxidase (HRP), alkaline phosphatase (AP), β-galactosidase, glucose oxidase, and the like, as well as various proteases. Other labels include, but are not limited to, fluorophores, dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837, 3,850,752, 3,939,350, 3,996,345, 4,277,437, 4,275,149, and 4,366,241. Many labels are commercially available and can be used in methods and assays of the disclosure, including detectable enzyme/substrate combinations (Pierce, Rockford IL; Santa Cruz Biotechnology, Dallas TX; Life Technologies, Carlsbad CA). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signal are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-gal actopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl) umbelliferyl phosphate for alkaline phosphatase; 4-methylumbelliferyl phosphate bis (2-amino- 2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase; 4-methylumbelliferyl β-D-galactopyranoside, fluorescein di((β-D-galactopyranoside) and naphthofluorescein di-(β-D-galactopyranoside) for β-galactosidase; 3-acetylumbelliferyl β-D-glucopyranoside and 4-methylumbelliferyl-β- D-glucopyranoside for β-glucosidase; and 4-methylumbelliferyl-α- D-galactopyranoside for α-galactosidase. Exemplary enzymes and substrates for producing a detectable signal are also described, for example, in U.S. Publication No. 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵²Sm, ¹⁵³Eu, ¹⁵⁴Sm, ¹⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Yb, ¹⁷²Yb, ¹⁷³Yb, ¹⁷⁴Yb, ¹⁷⁵Lu, and ¹⁷⁶Yb. Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other, and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

The methods of detecting a target protein disclosed herein can be used for concurrent or sequential detection of multiple target proteins in the same sample. For example, in some embodiments, the method comprises detecting two or more target proteins in the same sample. In some embodiments, the method comprises detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 96, 97, 98, 99, 100, or more different target proteins in the same sample. For example, in some embodiments, the method comprises detecting from 1 to 100 different proteins in the sample. In some embodiments, the method comprises detecting from 1 to 50 different proteins in the sample. Embodiments in which higher numbers of proteins are detected in the same sample may involve the use of cleavable labels, which are further described below. In the case of using fluorophores as labels, the fluorophores to be used for detection of multiple target proteins are selected so that each of the fluorophores are distinguishable and can be detected concurrently in a fluorescence microscope. Such fluorophores are selected to have spectral separation of the emissions so that distinct labeling of the target proteins can be detected concurrently. Methods of selecting suitable distinguishable fluorophores for use in methods of the disclosure are well known in the art (see, e.g., Johnson and Spence, “Molecular Probes Handbook, a Guide to Fluorescent Probes and Labeling Technologies, 11th ed., Life Technologies (2010)).

The label can be designed such that the labels are optionally cleavable. As used herein, a “cleavable label” refers to a label that is attached or conjugated to a label probe so that the label can be removed, for example, in order to use the same label in a subsequent round of labeling and detecting of targets. Methods for multiplex detection of nucleic acids of using cleavable labels have been described, e.g., in WO 2020/168162, which is incorporated herein by reference in its entirety, and are commercially available as RNAscope® HiPlex assays (e.g., RNAscope® HiPlex and RNAscope® HiPlex v2).

Generally, the labels are conjugated to the label probe by a chemical linker that is cleavable. Methods of conjugating a label to a label probe so that the label is cleavable are well known to those skilled in the art (see, e.g., Hermanson, Bioconjugate Techniques, Academic Press, San Diego (1996); Daniel et al., BioTechniques 24(3):484-489 (1998)). One particular system of labeling oligonucleotides is the FastTag™ system (Daniel et al., supra, 1998; Vector Laboratories, Burlingame CA). Various cleavable moieties can be included in the linker so that the label can be cleaved from the label probe. Such cleavable moieties include groups that can be chemically, photochemically, or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xa (cleavage site IEGR/), tobacco etch virus protease (cleavage site ENLYFQ/G), and thrombin (cleavage site LVPR/GS) (see, e.g., Oxford Genetics, Oxford, UK). Another cleavable moiety can be, for example, uracil-DNA (DNA containing uracil), which can be cleaved by uracil-DNA glycosylase (UNG) (see, e.g., Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

Step (iii) of the disclosed method comprises detecting a signal from the signal-generating complex. Well-known methods such as microscopy, cytometry (e.g., mass cytometry, cytometry by time of flight (CyTOF), flow cytometry), or spectroscopy can be utilized to detect chromogenic, fluorescent, or metal detectable signals associated with the respective targets. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of protein targets in the same sample.

The biological sample used in the disclosed methods can be derived from various sources. In one embodiment, the biological sample is a tissue specimen or is derived from a tissue specimen. In one embodiment, the biological sample is a blood sample or is derived from a blood sample. In one embodiment, the biological sample is a cytological sample or is derived from a cytological sample. In one embodiment, the biological sample is cultured cells. In another embodiment, the biological sample is a sample containing exosomes.

Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a target that is a biomarker indicative of a pathology.

The biological sample can be obtained from a subject, including a sample of biological tissue or fluid origin that is collected from an individual or some other source of biological material such as biopsy, autopsy, or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions, cells, and/or exosomes isolated from an organism such as a mammal. Exemplary biological samples include, but are not limited to, a cell culture, including a cell, a primary cell culture, a cell line, a tissue, an organ, an organoid, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, e.g., Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, e.g., Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In particular embodiments, the sample is a tissue specimen or is derived from a tissue specimen. In some embodiments, the tissue specimen is a formalin-fixed paraffin-embedded (FFPE) sample. In other particular embodiments, the sample is a blood sample or is derived from a blood sample. In still other particular embodiments, the sample is a cytological sample or is derived from a cytological sample. In some embodiments, the tissue specimen is fresh frozen. In some embodiments, the tissue specimen is prepared with a fixative. In some embodiments, the tissue specimen is prepared with a crosslinking fixative. In some embodiments, the fixative is neutral-buffered formalin (NBF), formaldehyde, glutaraldehyde, glyoxal, acrolein, osmium tetroxide, a permanganate fixative (e.g., potassium permanganate), a dichromate fixative (e.g., potassium dichromate), chromic acid, and a mixture of any thereof. In particular embodiments, the crosslinking agent is NBF, such as about 1% to about 20% NBF (e.g., 10% NBF).

In some embodiments, the method further comprises steps to prepare a sample for detection of the target. For example, if the sample is a FFPE sample, a de-paraffinization step can be used to remove paraffin and rehydrate the sample. In some embodiments, the method further comprises dehydrating the biological sample. In certain embodiments, the dehydration is carried out with ethanol of increasing concentrations, such as in the order of 70%, 95%, and 100% ethanol.

In some embodiments, the method further comprises an epitope retrieval step, where certain epitope retrieval buffer(s) can be added to unmask the target. In some embodiments, the epitope retrieval step comprises heating the sample. In some embodiments, the epitope retrieval step comprises heating the sample to about 50° C. to about 100° C. In one embodiment, the epitope retrieval step comprises heating the sample to about 88° C. Detergents (e.g., Triton X-100 or SDS) and Proteinase K can be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the targets. Optimal concentrations and durations of treatment can be experimentally determined as is well known in the art.

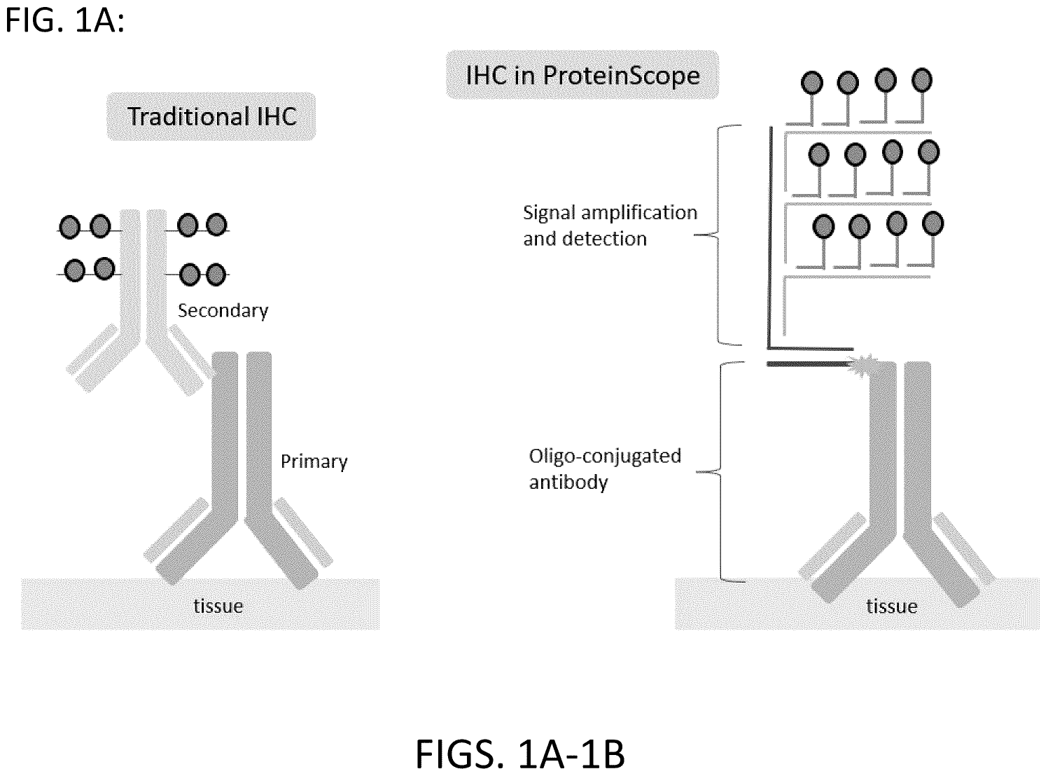

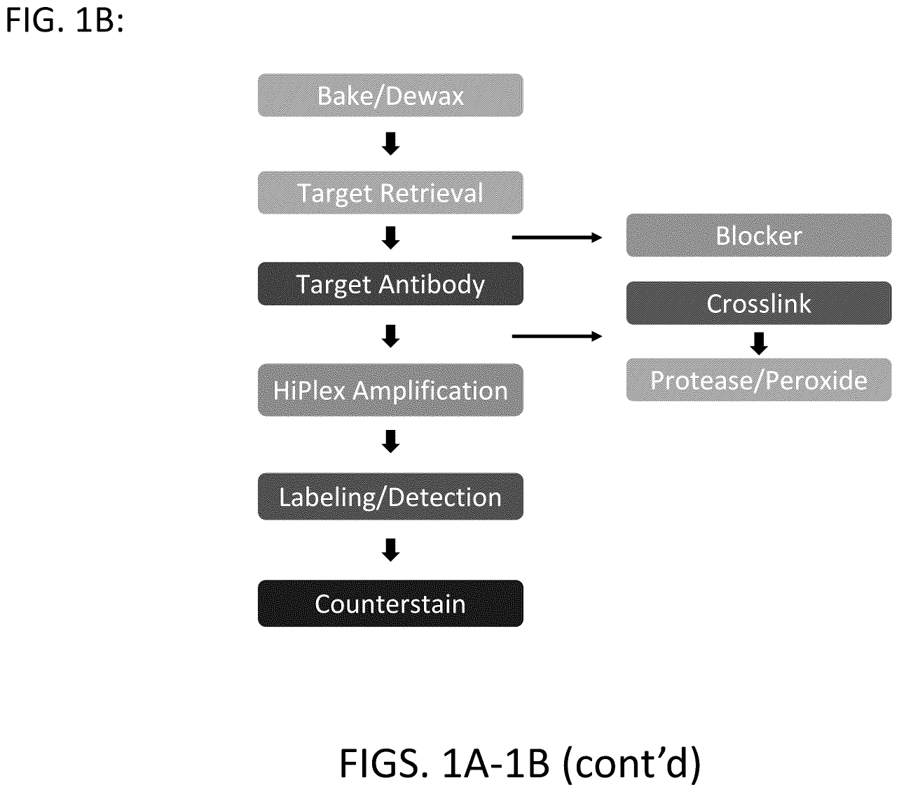

In some embodiments of the disclosed methods, an aptamer can be used in place of an antibody or fragment thereof. As used herein, the term “aptamer” refers to a single-stranded nucleic acid molecule (DNA or RNA) that can selectively bind to a specific target molecule, such as a target protein. In such embodiments, the oligo-conjugated antibody shown in the right panel of FIG. 1A would be replaced with an aptamer. Accordingly, provided herein is a method for detecting a target protein in a biological sample, comprising: (i) contacting the sample with an aptamer, wherein the aptamer is covalently attached to an oligonucleotide; (ii) contacting the sample with a signal-generating complex, wherein the signal-generating complex comprises a nucleic acid component capable of hybridizing to the oligonucleotide; and (iii) detecting a signal from the signal-generating complex. In some embodiments, the aptamer has a length of about 10 nucleotides to about 100 nucleotides, or a length of about 20 nucleotides to about 60 nucleotides. In some embodiments, one of the more nucleotides in the aptamer is a modified nucleotide. As discussed above for the antibody-oligonucleotide conjugates, the aptamer can be directly attached to the oligonucleotide via a covalent bond. In such embodiments, the aptamer-oligonucleotide conjugate will comprise a single polynucleotide sequence, with one portion corresponding to the aptamer that binds to the target protein, and one portion corresponding to a sequence that can hybridize to a nucleic acid component of the signal-generating complex. In other embodiments, the aptamer sequence and the oligonucleotide sequence are separated by a linker, such as any linker disclosed herein.

Embodiments of the present disclosure also include a method for detecting a target protein in a biological sample. In accordance with these embodiments, the method includes (i) contacting the sample with at least one antibody or a fragment thereof, wherein the at least one antibody or the fragment thereof is covalently attached to an oligonucleotide; (ii) contacting the sample with a signal-generating complex, wherein the signal-generating complex comprises a nucleic acid component capable of hybridizing to the oligonucleotide; and (iii) detecting a signal from the signal-generating complex. In some embodiments, the method includes contacting the sample with a crosslinking agent after step (i) and before step (ii).

In some embodiments, the crosslinking agent is glutaraldehyde, or a derivative thereof. In some embodiments, the method comprises contacting the sample with glutaraldehyde, or a derivative thereof, after step (i) and before step (ii). In some embodiments, the method comprises using glutaraldehyde at a concentration that ranges from about 0.001% to about 5%. In some embodiments, the method comprises using glutaraldehyde at a concentration that ranges from about 0.001% to about 2.5%. In some embodiments, the method comprises using glutaraldehyde at a concentration that ranges from about 0.001% to about 1%. In some embodiments, the method comprises using glutaraldehyde at a concentration that ranges from about 0.001% to about 0.5%. In some embodiments, the method comprises using glutaraldehyde at a concentration that ranges from about 0.001% to about 0.01%. In some embodiments, the method comprises using glutaraldehyde at a concentration that ranges from about 0.01% to about 5%. In some embodiments, the method comprises using glutaraldehyde at a concentration that ranges from about 0.1% to about 5%. In some embodiments, the method comprises using glutaraldehyde at a concentration that ranges from about 0.5% to about 5%. In some embodiments, the method comprises using glutaraldehyde at a concentration that ranges from about 1.0% to about 5%. In some embodiments, the method comprises using glutaraldehyde at a concentration that ranges from about 2.5% to about 5%. In some embodiments, the method comprises using glutaraldehyde at any of these concentrations in 10% neutral buffered formalin (NBF) or any similar fixative solution. In some embodiments, the method comprises using glutaraldehyde at any of these concentrations in 15% NBF, 14% NBF, 13% NBF, 12% NBF, 11% NBF, 10% NBF, 9% NBF, 8% NBF, 7% NBF, 6% NBF, 5% NBF, 4% NBF, 3% NBF, 152 NBF, or 1% NBF.

In accordance with the embodiments, the at least one antibody or the fragment thereof comprises a primary antibody specific for an antigen on the target protein. In some embodiments, the at least one antibody or fragment thereof comprises at least two primary antibodies specific for different antigens on different target proteins (e.g., FIG. 12A). In some embodiments, the at least one antibody or fragment thereof comprises at least two primary antibodies specific for different antigens on the same target protein. In some embodiments, the at least one primary antibody or the at least two primary antibodies are covalently attached to the oligonucleotide.

In some embodiments, the at least one antibody or fragment thereof comprises at least one secondary antibody specific for an antigen on at least one primary antibody, wherein the at least one primary antibody is specific for an antigen on the target protein. In some embodiments, the at least one antibody or fragment thereof comprises at least two secondary antibodies specific for different antigens on at least two primary antibodies, wherein the at least two primary antibodies are specific for different antigens on different target proteins (e.g., FIG. 12A). In some embodiments, the at least one antibody or fragment thereof comprises at least two secondary antibodies specific for different antigens on at least two primary antibodies, wherein the at least two primary antibodies are specific for different antigens on the same target protein. In some embodiments, the at least one secondary antibody or the at least two secondary antibodies is/are covalently attached to the oligonucleotide.

In some embodiments, the method includes multiplexed detection of a plurality of target proteins. For example, in some embodiments, the method comprises (i) contacting the sample with a plurality of antibodies or fragments thereof, wherein the plurality of antibodies or fragments thereof comprises at least one primary antibody specific for a target protein and wherein the at least one primary antibody is covalently attached to an oligonucleotide; (ii) contacting the sample with a plurality of pre-amplifiers comprising binding sites for the oligonucleotide and a plurality of binding sites for an amplifier; (iii) contacting the sample with a plurality of amplifiers comprising binding sites for the pre-amplifiers and binding sites for label probes; (iv) contacting the sample with a plurality of label probes comprising a label and binding sites for the amplifiers, wherein the labels are cleavable; and (v) detecting the plurality of label probes, thereby detecting the target proteins. As described further herein, in some embodiments, the method further comprises cleaving the labels from the plurality of label probes; (vi) contacting the sample with at least a second plurality of label probes comprising a label and binding sites for the amplifiers; and (xi) detecting the second plurality of label probes, thereby detecting a second target protein.

In some embodiments, the method comprises (i) contacting the sample with a plurality of antibodies or fragments thereof, wherein the plurality of antibodies or fragments thereof comprises at least one primary antibody specific for a target protein and at least one secondary antibody specific for the primary antibody, and wherein the at least one secondary antibody is covalently attached to an oligonucleotide; (ii) contacting the sample with a plurality of pre-amplifiers comprising binding sites for the oligonucleotide and a plurality of binding sites for an amplifier; (iii) contacting the sample with a plurality of amplifiers comprising binding sites for the pre-amplifiers and binding sites for label probes; (iv) contacting the sample with a plurality of label probes comprising a label and binding sites for the amplifiers, wherein the labels are cleavable; and (v) detecting the plurality of label probes, thereby detecting the target protein. As described further herein, in some embodiments, the method further comprises cleaving the labels from the plurality of label probes; (vi) contacting the sample with at least a second plurality of label probes comprising a label and binding sites for the amplifiers; and (xi) detecting the second plurality of label probes, thereby detecting a second target protein.

c. Methods of Detecting a Target Nucleic Acid and a Target Protein

In another aspect, provided herein is a method for detecting a target nucleic acid and a target protein in a biological sample, comprising: (i) contacting the sample with an antibody or a fragment thereof, wherein the antibody or the fragment thereof is covalently attached to an oligonucleotide; (ii) contacting the sample with a first signal-generating complex, wherein the first signal-generating complex comprises a nucleic acid component capable of hybridizing to the oligonucleotide; (iii) detecting a signal from the first signal-generating complex to thereby detect the target protein in the sample; and (iv) detecting the target nucleic acid by in situ hybridization.

Steps (i), (ii), and (iii) are analogous to those discussed above for the methods of detecting a protein in a sample. Any of the aspects or embodiments disclosed above in section b (“Methods of Detecting a Target Protein”) are also applicable to steps (i), (ii), and (iii) of this co-detection method. Optional additional steps disclosed in section b are also applicable to this method; e.g., use of blocking agent prior to step (i), use of a crosslinking agent after step (i) and before step (ii), use of a protease after treating the biological sample with the crosslinking agent and before step (ii), etc.

This method further comprises an ISH step to detect a target nucleic acid in the sample. Methods for in situ detection of nucleic acids are well-known to those skilled in the art (see, e.g., US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, in a sample, in particular a portion or section of tissue or cells (in situ). The probe types can be double stranded DNA (dsDNA), single stranded DNA (ssDNA), single stranded complementary RNA (sscRNA), and/or synthetic oligonucleotides. In some specific embodiments, the ISH used herein is RNAscope®, which is generally described above in section b.

In some embodiments, the ISH step of the disclosed methods comprises: (a) providing at least one set of one or more target probe(s) capable of hybridizing to said target nucleic acid; (b) providing a signal-generating complex capable of hybridizing to said set of one or more target probe(s), said signal-generating complex comprises a nucleic acid component capable of hybridizing to said set of one or more target probe(s) and a label probe; (c) hybridizing said target nucleic acid to said set of one or more target probe(s); and (d) capturing the signal-generating complex to said set of one or more target probe(s) and thereby capturing the signal-generating complex to said target nucleic acid.

As used herein, a “target probe” is a polynucleotide that is capable of hybridizing to a target nucleic acid and capturing or binding a label probe or signal-generating complex (SGC) component to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. The target probe is generally single stranded so that the complementary sequence is available to hybridize with a corresponding target nucleic acid, label probe, amplifier, pre-amplifier, or pre-pre-amplifier. In some embodiments, the target probes are provided as a pair.

In some embodiments, each set of one or more target probe(s) comprises a single probe. In other embodiments, each set of one or more target probe(s) comprises two probes. In yet other embodiments, each set of one or more target probe(s) comprises more than two probes.

In some embodiments, when each set of target probes comprises a single target probe, a signal-generating complex is formed when the single target probe is bound to the target nucleic acid. In other embodiments, when each set of target probes comprise two target probes, a signal-generating complex is formed when both members of a target probe pair are bound to the target nucleic acid.

In some embodiments, each target probe comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, one set of one or more target probe(s) is used to detect a target nucleic acid. In other embodiments, two or more sets of one or more target probe(s) are used to detect a target nucleic acid. In some embodiments, two sets of one or more target probe(s) are used to detect a target nucleic acid. In some embodiments, three sets of one or more target probe(s) are used to detect a target nucleic acid. In some embodiments, four sets of one or more target probe(s) are used to detect a target nucleic acid. In some embodiments, five sets of one or more target probe(s) are used to detect a target nucleic acid. In some embodiments, six sets of one or more target probe(s) are used to detect a target nucleic acid. In some embodiments, seven sets of one or more target probe(s) are used to detect a target nucleic acid. In some embodiments, eight sets of one or more target probe(s) are used to detect a target nucleic acid. In some embodiments, nine sets of one or more target probe(s) are used to detect a target nucleic acid. In some embodiments, ten sets of one or more target probe(s) are used to detect a target nucleic acid. In some embodiments, more than 10 sets of one or more target probe(s) are used to detect a target nucleic acid. In some embodiments, more than 15 sets of one or more target probe(s) are used to detect a target nucleic acid. In some embodiments, more than 20 sets of one or more target probe(s) are used to detect a target nucleic acid. In some embodiments, more than 30 sets of one or more target probe(s) are used to detect a target nucleic acid.

In some embodiments, the SGC for detecting the target nucleic acid comprises additional components such an amplifier, a pre-amplifier, and/or a pre-pre-amplifier. The terms “label probe,” “amplifier,” “pre-amplifier,” and “pre-pre-amplifier” are defined above in section b, and the same definitions apply in relation to this method. In the context of methods of detecting a target nucleic acid, it will be understood that the “target” referenced in those definitions can be the target nucleic acid, or the target probe that hybridizes to the target nucleic acid. For example, the amplifier can hybridize to at least one target probe of a pair of target probes, to both target probes of a pair of target probes, or to nucleic acid bound to a target probe such as an amplifier, pre-amplifier or pre-pre-amplifier; the pre-amplifier serves as an intermediate binding component between one or more target probes and one or more amplifiers, typically hybridizing simultaneously to one or more target probes and to a plurality of amplifiers; and the pre-pre-amplifier serves as an intermediate binding component between one or more target probes and one or more pre-amplifiers, typically hybridizing simultaneously to one or more target probes and to a plurality of pre-amplifiers.

The labels used to detect the target nucleic acids can be the same as those described in section b for detection of target proteins. In the disclosed co-detection methods, the labels are selected such that each is distinguishable and can be detected concurrently. For example, in the case of using fluorophores as labels, the fluorophores to be used for detection of multiple targets are selected so that each of the fluorophores are distinguishable and can be detected concurrently in a fluorescence microscope. Such fluorophores are selected to have spectral separation of the emissions so that distinct labeling of the target nucleic acids can be detected concurrently. Methods of selecting suitable distinguishable fluorophores for use in methods of the disclosure are well known in the art and are discussed above in section b.

In some embodiments, the nucleic acid detected by the methods can be any nucleic acid present in the sample. In some embodiments, the target nucleic acid is RNA. In some embodiments, the RNA is selected from messenger RNA (mRNA), microRNA (miRNA), a pre-miRNA, ribosomal RNA (rRNA), mitochondrial RNA, non-coding RNA, a circular RNA, a small interfering RNA (siRNA), a guide RNA, or an antisense oligonucleotide. In some embodiments, the target nucleic acid is DNA.

In some embodiments, the method provided herein is for detecting multiple target nucleic acids in the sample. For example, in some embodiments, the method comprises detecting 2, 3, 4, 5, 6, 7, 8, 9, 10, 11, 12, 13, 14, 16, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different target nucleic acids in the same sample. For example, in some embodiments, the method comprises detecting from 1 to 100 different target nucleic acids in the sample. In some embodiments, the method comprises detecting from 1 to 50 different target nucleic acids in the sample. Embodiments in which higher numbers of proteins are detected in the same sample may involve the use of cleavable labels, which are further described below.

In some embodiments, the method provided herein is for detecting multiple target proteins and multiple target nucleic acids in the sample. For example, in some embodiments, the method comprises detecting 2, 3, 4, 5, 6, 7, 8, 9, 10, 11, 12, 13, 14, 16, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different target proteins in the same sample and 2, 3, 4, 5, 6, 7, 8, 9, 10, 11, 12, 13, 14, 16, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different target nucleic acids in the same sample.

There are significant potential benefits to the disclosed methods of detecting a target protein and a target nucleic acid in the same biological sample. Such methods can increase the throughput of analysis and reduce the burden of time and cost with investigating the individual components in separate sample. Combined detection in the same sample can also provide information that cannot be discerned from different samples, such as visualizing both a secreted protein and the origin of its cell(s), correlating RNA and protein expression, examining cell-type specific gene expression, validating antibodies, discriminating antibody detection of multiple targets (e.g., different transcript variants or highly homologous targets, and interaction of distinct cell types.

d. Kits

In yet another aspect, provided herein is a kit for performing the various methods described herein, including the methods of detecting a target protein disclosed in section b, and the methods of detecting a target protein and a target nucleic acid disclosed in section c.

In one aspect, provided herein is a kit for detecting a target protein in a biological sample, comprising: (i) an antibody or a fragment thereof, wherein the antibody or the fragment thereof is covalently attached to an oligonucleotide; and (ii) a signal-generating complex, wherein the signal-generating complex comprises a nucleic acid component capable of hybridizing to the oligonucleotide.

In another aspect, provided herein is a kit for detecting a target protein in a biological sample, comprising: (i) an oligonucleotide comprising a reactive moiety for conjugation to an antibody; and (ii) a signal-generating complex, wherein the signal-generating complex comprises a nucleic acid component capable of hybridizing to the oligonucleotide. In some embodiments of such kits, the kit further comprises a conjugation reagent for conjugating the oligonucleotide to an antibody (e.g., an antibody supplied separately from the kit).

In another aspect, provided herein is a kit for detecting a target nucleic acid and a target protein in a biological sample, comprising: (i) an antibody or a fragment thereof, wherein the antibody or the fragment thereof is covalently attached to an oligonucleotide; (ii) a first signal-generating complex, wherein the signal-generating complex comprises a nucleic acid component capable of hybridizing to the oligonucleotide; (iii) one or more target probe(s) capable of hybridizing to the target nucleic acid; and (iv) a second signal-generating complex capable of hybridizing to the one or more target probe(s), wherein the second signal-generating complex comprises a nucleic acid component capable of hybridizing to the one or more target probe(s).

The antibody or the fragment thereof, which is covalently attached to an oligonucleotide, is as described above in section b. In the disclosed kit, the antibody or the fragment thereof, the oligonucleotide, and the optional linker can be any of those described above in section b. Similarly, the signal-generating complex(es) included in the kit can be any of those described above in section b or section c. In the kits that can include one or more target probe(s), the target probe(s) can be any of those described above in section c.

In some embodiments, the kit further comprises a blocking agent, a crosslinking agent, a protease, or any combination thereof. The blocking agent, the crosslinking agent, and the protease can be selected from any of those described herein in section b.

The kit may further comprise packaging material, which refers to a physical structure housing the components of the kit. The packaging material can maintain the components in a sterile environment, and can be made of material commonly used for such purposes (e.g., paper, corrugated fiber, glass, plastic, foil, ampules, vials, tubes, etc.).

Kits provided herein can include labels or inserts, which can include information on a condition, disorder, disease, or symptom for which the kit component may be used for. Labels or inserts can include instructions for carrying out any of the methods disclosed herein. In some embodiments, labels or inserts can include instructions for a clinician or for a subject to use one or more of the kit components in a method, treatment protocol, or therapeutic regimen.

In some embodiments, the kit provided herein is used for mapping spatial organization in a complex tissue. In some embodiments, the kit provided herein is used for identifying cell types and new cell types. In some embodiments, the kit provided herein is used for identifying cellular states. In other embodiments, the kit provided herein is used for identifying cell types and new cell types in a tumor microenvironment. In some embodiments, the kit provided herein is used for identifying cellular states in a tumor microenvironment.

In some embodiments, the kit provided herein is used for detecting altered gene expression in diseased cells and tissues. In some embodiments, the kit provided herein is used for localizing altered gene expression in specific cell types and understanding tumor heterogeneity. In some embodiments, the kit provided herein is used for studying tumor-immune cell interactions. In some embodiments, the kit provided herein is used for detecting biomarkers for cancer diagnosis and prognosis. In some embodiments, the kit provided herein is used for detecting therapeutic targets for cancer treatment. In some embodiments, the kit provided herein is used for facilitating the validation of novel antibodies.

Embodiments of the present disclosure also include a kit for detecting a target protein in a biological sample. In accordance with these embodiments, the kit includes (i) at least one antibody or a fragment thereof, wherein the at least one antibody or the fragment thereof is covalently attached to an oligonucleotide; and (ii) a signal-generating complex, wherein the signal-generating complex comprises a nucleic acid component capable of hybridizing to the oligonucleotide.

In some embodiments of the kit, the at least one antibody or the fragment thereof comprises a primary antibody specific for an antigen on the target protein. In some embodiments, the at least one antibody or fragment thereof comprises at least two primary antibodies specific for different antigens on different target proteins. In some embodiments, the at least one antibody or fragment thereof comprises at least two primary antibodies specific for different antigens on the same target protein. In some embodiments, the at least one primary antibody or the at least two primary antibodies are covalently attached to the oligonucleotide.

In some embodiments of the kit, the at least one antibody or fragment thereof comprises at least one secondary antibody specific for an antigen on at least one primary antibody, wherein the at least one primary antibody is specific for an antigen on the target protein. In some embodiments, the at least one antibody or fragment thereof comprises at least two secondary antibodies specific for different antigens on at least two primary antibodies, wherein the at least two primary antibodies are specific for different antigens on different target proteins. In some embodiments, the at least one antibody or fragment thereof comprises at least two secondary antibodies specific for different antigens on at least two primary antibodies, wherein the at least two primary antibodies are specific for different antigens on the same target protein. In some embodiments, the at least one secondary antibody or the at least two secondary antibodies is/are covalently attached to the oligonucleotide.

e. Image Processing

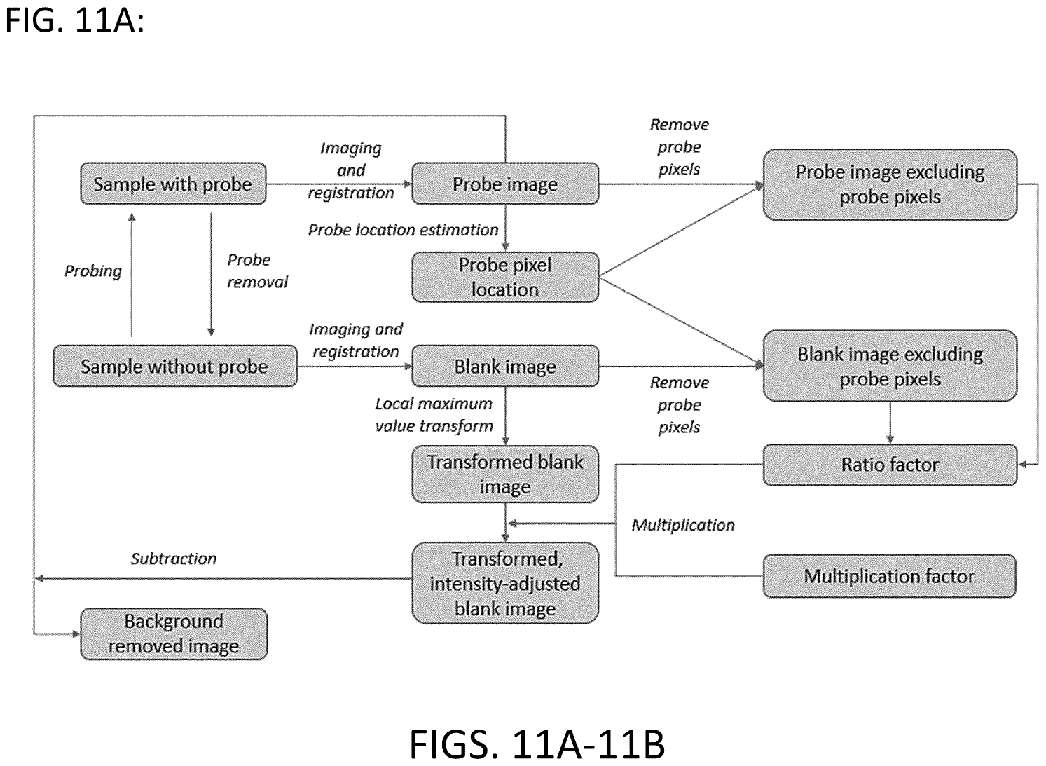

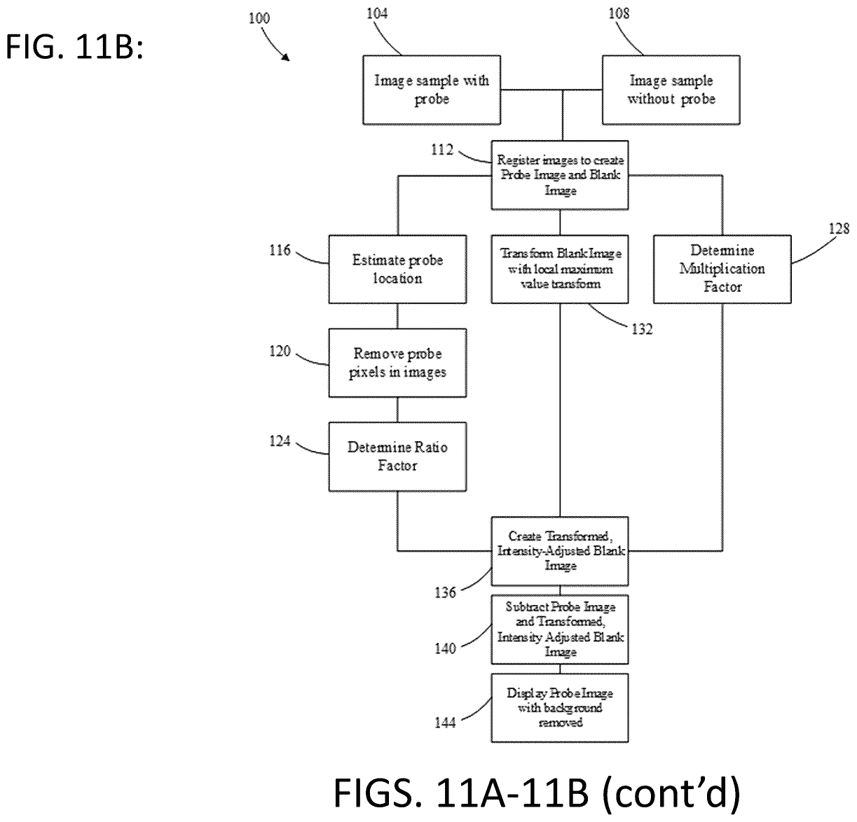

Embodiments of the present disclosure also include a method 100 for enhancing detection of a target. In some embodiments, the method 100 includes an image processing method. The method 100 is illustrated in FIG. 11B as a flowchart of steps, whereas FIG. 11A illustrates a plurality of images and corresponding factors used in the method 100 to modify the images. In the illustrated embodiment, the method 100 is implemented at least in part with a computer having corresponding instructions stored on a memory (i.e., a non-transitory computer readable medium). The final images, and in some embodiments the intermediate images, from the method 100 are stored in a memory. In some embodiments, the memory is accessible by a network. In some embodiments, user input or instructions are receivable or accessible over the network.

The method 100 includes imaging a sample with a target signal (STEP 104) to create a probe image and imaging a sample with no target signal (STEP 108) to create a background image (i.e., “blank image”). In some embodiments, the imaging utilizes a fluorescent microscope coupled to a computer via a network. In some embodiments, the target signal is obtained by subjecting the sample to a fluorescent in situ hybridization assay and/or an immunofluorescence assay. In some embodiments, the background image with no target signal is obtained by removing the target signal from the sample (i.e., by a cleaving process). In other embodiments, the background image with no target signal is obtained before the assay is performed. In other words, in some embodiments STEP 104 occurs before STEP 108 and in other embodiments STEP 104 occurs after STEP 108. In some embodiments, the target signal comprises a fluorescent label bound to a target nucleic acid. In other embodiments, the target signal comprises a fluorescent label bound to a target peptide or polypeptide.

With continued reference to FIG. 11B, the method 100 includes a STEP 112 of registering the probe image and the background image. Potential background fluorescence discrepancy between the probe image and the background image creates spatial pattern mismatches that occur due to whole sample movement between different rounds of image acquisition. To remove such discrepancies, image registration techniques (e.g., phase correlation) are utilized. Robust image registration (e.g., STEP 112) utilized detection and matching of image features to compensate for any global sample movement (i.e., translation and rotation).

The method 100 further includes modifying the background image (STEP 136) to create an adjusted background image (e.g., transformed, intensity-adjusted blank image) based on at least one image metric. As explained further herein, the at least one image metric is a ratio factor (STEPS 116, 120, 124), a multiplication factor (STEP 128), a local maximum value transform (STEP 132), and any other suitable metric. In some embodiments, the method 100 includes a single image metric. In other embodiments, the method 100 includes a combination of image metrics.

With continued reference to FIG. 11B, the method 100 further includes subtracting the adjusted background image from the probe image (STEP 140) to create a final image comprising an enhanced target signal. In other words, the modified (i.e., transformed, adjusted, scaled, etc.) blank image is used in the subtracting step (STEP 140) instead of the original blank image. In some embodiments, the enhanced target signal includes enhanced contrast. In some embodiments, the method 100 includes displaying the final image (STEP 144) on a display (e.g., a computer display). The final image may be saved to a memory and may be accessible by a user, for example, over a network. As such, the method 100 provides improved signal detection in the presence of a background with tissue autofluorescence.

In some embodiments, the image metric is a ratio factor to account for intensity differences in background between the blank image and the probe image. Intensity differences can occur when image acquisition settings are different or from photobleaching during fluorophore excitation. To compensate for background intensity differences, the method 100 includes STEPS 116, 120, and 124 to determine a ratio factor that compares the overall background intensity of the probe image versus the blank image. First, the pixel locations of the probe are estimated (STEP 116). The probe locations in the probe image are estimated using, for example, the White Top Hat algorithm (Gonzalez & Woods, 2008, Digital Image Processing), bandpass filtering (Shenoi, 2006, Introduction to Digital Signal Processing and Filter Design), or any combination of suitable methods. After determining an estimated location of the target signals in the probe image (STEP 116), the pixels at the estimated probe locations are excluded from both the probe image and the blank image (STEP 120), resulting in background-pixel-only images (i.e., background-only images). In other words, STEP 120 includes removing the estimated location from the probe image to create a first background-only image and removing the estimated location from the blank image (background image) to create a second background-only image.

Following removal of the estimated probe locations from both images (STEP 120), the method 100 includes a STEP 124 to determine a ratio factor. In other words, a statistical metric for both the probe-excluded blank image and the probe-excluded probe image is evaluated and incorporated into a ratio factor. As explained further herein, the ratio factor is utilized in some embodiments to modify the background image to create an adjusted background image (STEP 136). In other words, modifying the background image to create an adjusted background image can include, in some embodiments, scaling the background image by the ratio factor.

In some embodiments, the at least one image metric is a ratio factor of the first background-only image and the second background-only image. For example, the ratio factor in some embodiments is a first intensity to a second intensity, with the first intensity is determined from the first background-only image and the second intensity is determined from the second background-only image. In some embodiments, the first and second intensities used in the ratio factor are statistical metrics such as a statistical mean, median, or a combination of both for any portion of (including all) the intensity values in an image.

In some embodiments, the first intensity is the mean of a plurality of pixel intensity values in the first background-only image and the second intensity is the mean of a plurality of pixel intensity values in the second background-only image. In some embodiments, the mean is of all the pixel intensity values in the image. In other embodiments, the first intensity is the median of a plurality of pixel intensity values in the first background-only image, and the second intensity is the median of a plurality of pixel intensity values in the second background-only image. In some embodiments, the median is of all the pixel intensity values in the image. In another embodiment, the first intensity is the mean of a central approximately 80% of all the pixel intensity values (i.e., excluding the approximate top 10% and the approximate bottom 10%) in the first background-only image, and the second intensity is the mean of a central approximately 80% of all the pixel intensity values in the second background-only image.

In some embodiments, the image metric is a multiplication factor to account for potential local intensity differences between the blank image and the probe image. In particular, the method 100 in the illustrated embodiment includes STEP 128 to determine the multiplication factor. In some embodiments, the multiplication factor is within a range of approximately 1.0 to approximately 1.2. In other embodiments, the multiplication factor is within a range of approximately 1.0 to approximately 1.1. As explained further herein, the multiplication factor is utilized in some embodiments to modify the background image to create an adjusted background image (STEP 136). In other words, modifying the background image to create an adjusted background image can include, in some embodiments, scaling the background image by the multiplication factor.

In some embodiments, the image metric is a local maximum value transform. In particular, the method 100 in the illustrated embodiment includes STEP 132 to transform the blank image with a local maximum value transform. Even after global image registration, there may remain local background pattern mismatches that from, for example, image acquisition at different focal planes, or samples not firmly attached to the supporting material (e.g., glass slides) and partially moving between imaging sessions. To resolve this issue, local mismatches are compensated with a transform. In the illustrated embodiment, for each pixel in the blank image (“pixel of interest”), a neighborhood of a pre-defined radius surrounding the pixel of interest is searched. The search process will find the pixel of maximum intensity, and this maximum intensity is assigned to that pixel of interest. This searching procedure is performed for each pixel of interest, searching its neighborhood in the original blank image, to form a transformed blank image. As explained in greater detail herein, the transformed blank image can be used instead of the original blank image in the later subtracting step (i.e., STEP 140). In some embodiments, the pre-defined radius (“match distance”) is adjustable.

In some embodiments, the pre-defined radius used in the local maximum valve transform is within a range of approximately 0 to approximately 5 pixels. In other words, the local maximum value transform includes a search radius within a range of approximately 0 to 5 pixels. A pre-defined radius of 0 pixels is utilized, for example, when there is no noticeable local background pattern mismatch. In some embodiments, the search area is simplified to reduce computational time by using eight angularly equally spaced lines (i.e., 45 degrees apart), each with a single-pixel width, radiating from the pixel of interest.

In some embodiments, the image metric is a block-matching transform. In particular, the method 100, in some embodiments, includes a step to transform the blank image with a block-matching transform. In some embodiments, the block-matching transform is used in place of the local maximum value transform to resolve the issue of local mismatches. In some embodiments, a block (“block of interest”) is used with a pre-defined block size (e.g., a 3-pixel-by-3-pixel block). Each block in the blank image is compared with blocks of the same size in the probe image in nearby locations (i.e., within a pre-defined block search size). The search determines the nearby block that is most similar to the block of interest. A similarity metric is utilized to measure the similarity of the blocks, and the searched nearby block with the highest similarity metric is determined to be the target block. Then, the block of interest is moved to the corresponding location of the target block. In some embodiments, the similarity metric is a mean absolute difference, a sum of absolute difference, a mean squared difference, or a sum of squared difference, wherein the differences are the pixel intensity differences between the two blocks being compared. As such, the block-matching transform is performed for each block of interest, searching its corresponding neighborhood in the probe image and moving its location accordingly, to form a transformed blank image. In some embodiments, this transformed blank image is used instead of the original blank image in later subtracting steps (i.e., STEP 140).

In some embodiments, the pre-defined block size and the pre-defined block search size are adjustable. In some embodiments, the pre-defined block size used in the block-matching transform is within a range of approximately 1 to approximately 10 pixels. In other words, the block-matching transform includes a block size within a range of approximately 1 to 10 pixels. In some embodiments, the pre-defined block search size used in the block matching transform is within a range of approximately 1 to approximately 10 pixels. In other words, the block-matching transform includes a block search size within a range of approximately 1 to 10 pixels.

In some embodiments, the method for enhancing detection of a target includes any combination of the steps described herein, in various orders. In some embodiments, steps may be omitted. Further, the order of the steps may be reversed, altered, or performed simultaneously.

In at least one embodiment, the electronic-based aspects of the method 100 may be implemented in software (e.g., stored on non-transitory computer-readable medium) executable by a computer with one or more processing units, such as a microprocessor and/or application specific integrated circuits (“ASICs”). Some embodiments may include hardware, software, and electronic components or modules. As such, it should be noted that a plurality of hardware and software-based devices, as well as a plurality of different structural components, may be utilized to implement the embodiments.

### EXAMPLES

The following is a description of methods, materials, and results corresponding to the various embodiments of the present disclosure. These descriptions are provided as examples and are not intended to be limiting. Rather, these examples are intended to provide those of ordinary skill in the art with a description of how to make and use the various embodiments of the present disclosure. These examples are not intended to limit the scope of what the inventors regard as inventive subject matter, nor are they intended to represent all of the experiments that can be performed. It is to be understood that exemplary descriptions written in the present tense were not necessarily performed, but rather that the descriptions can be performed to generate the data associated with the teachings of the present disclosure. Efforts have been made to ensure accuracy with respect to numbers used (e.g., amounts, percentages, etc.), but some experimental error and deviation may be present.

Example 1

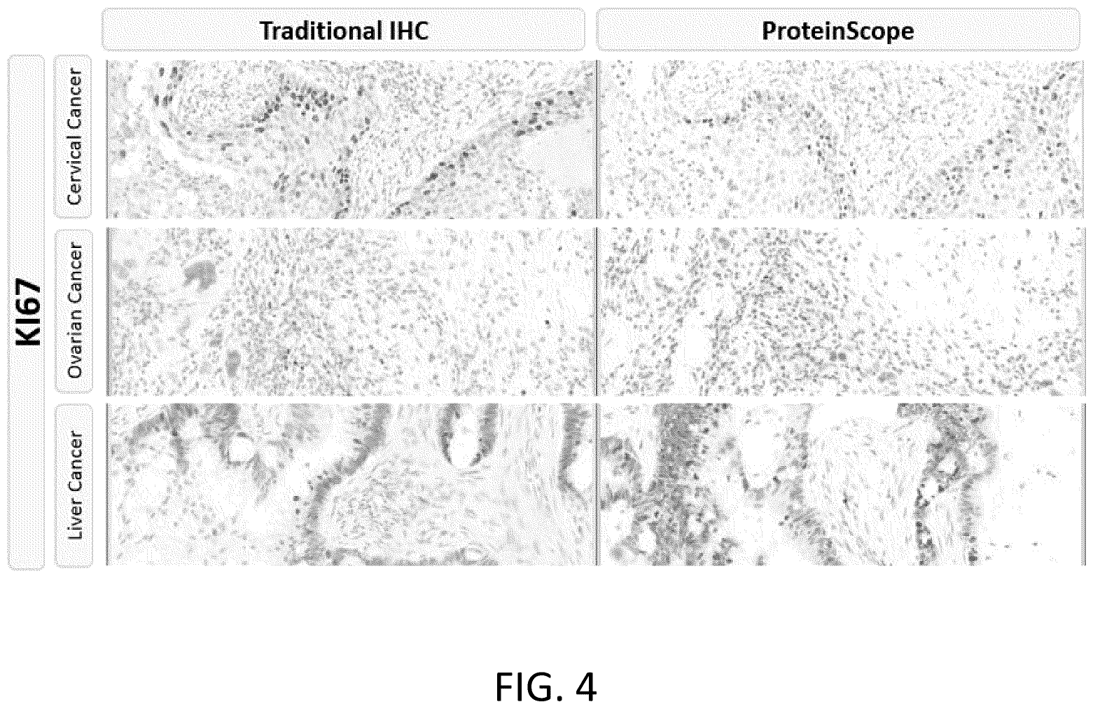

To demonstrate that a protein target can be detected with the methods of the present disclosure (e.g., ProteinScope), a workflow was developed for chromogenic detection of a single protein target (FIG. 1B). In this workflow, ProteinScope is automated using the Leica Biosystems BOND Rx automated staining instrument, using a primary antibody conjugated to an oligonucleotide using Sulfo-SMCC crosslinking. Antibody-bound oligonucleotide is detected using a signal generating complex, formed using the RNAscope® HiPlex12 Reagents Kit v2 in the T1 channel and detected using an HRP-conjugated label probe and DAB chromogen from the Leica BOND Polymer Refine Detection Kit. The resulting detection can be compared to traditional IHC detection on distinct sections from the same FFPE tissue samples, stained on the Leica Bond Rx automated staining instrument using unconjugated primary antibody and leveraging secondary detection and DAB chromogen from the Leica BOND Polymer Refine Detection Kit. In one example, traditional IHC using the monoclonal anti-human KI67 antibody (R&D Systems, clone 1297A) was compared to ProteinScope using the same clone, conjugated to an oligonucleotide using Sulfo-SMCC crosslinking. Nuclear staining consistent with KI67 detection was observed with both traditional IHC and ProteinScope methods (FIG. 4).

Example 2

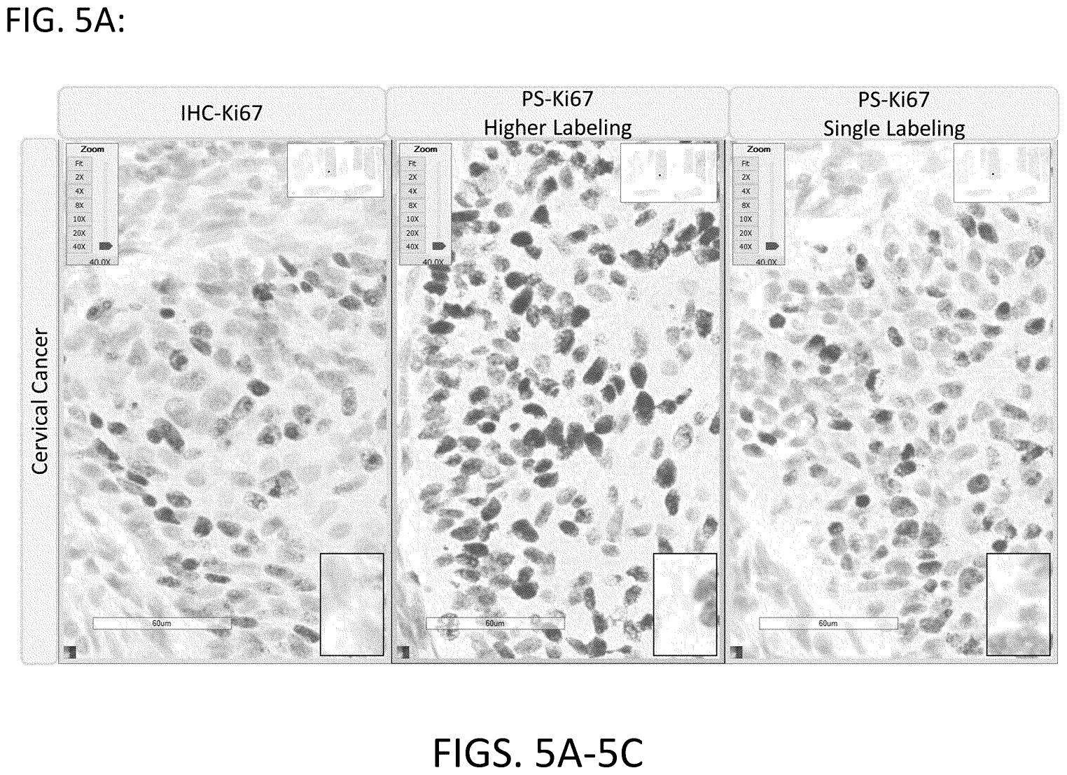

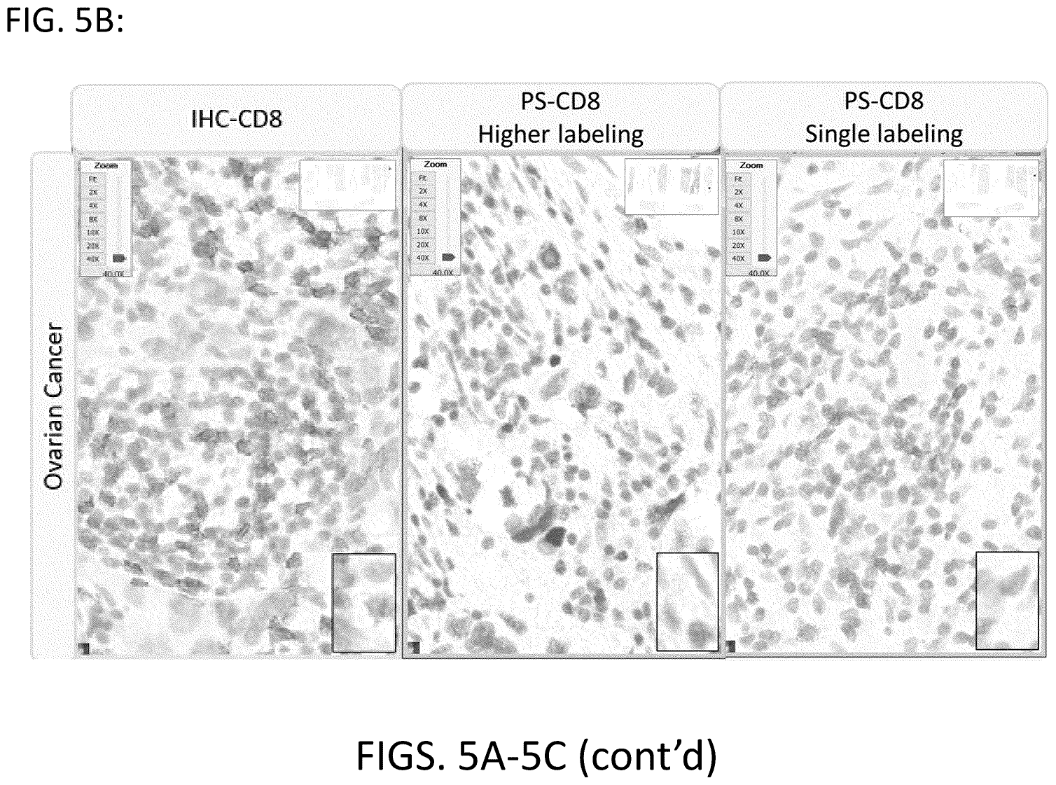

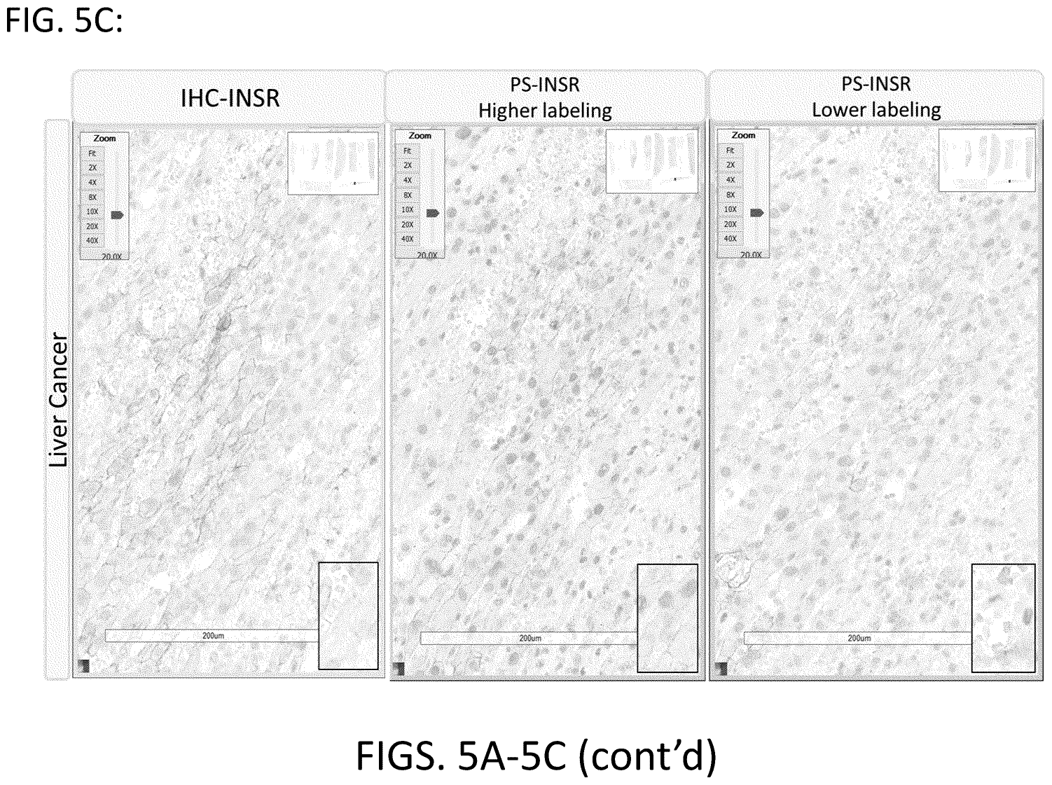

Experiments were conducted to test the impact of the number of oligonucleotides conjugated per antibody. Generally, higher amounts of labelling allows for the generation of multiple signal generating complexes and higher overall amplification, while use of single labeling allows for greater consistency and standardization of target amplification level. For multiple protein targets-of-interest, primary antibodies with various degrees of labelling (oligo/antibody ratio) allowed for successful tissue staining, as compared to IHC (KI67—FIG. 5A; CD8—FIG. 5B; INSR—FIG. 5C). For each set of experiments, traditional IHC was performed on FFPE sections of cancer tissues on the Leica Biosy stems BOND Rx automated staining instrument using HRP Polymer secondary antibody and DAB chromogen from the Leica BOND Polymer Refine Detection Kit. For ProteinScope, the primary antibodies were each conjugated to oligonucleotides by Sulfo-SMCC crosslinking and conjugates with different degrees of labelling were separated by size exclusion chromatography. To compare target protein detection with IHC, ProteinScope was performed on distinct sections from the same FFPE cancer samples on the Leica Biosystems BOND Rx automated staining instrument, and ProteinScope staining was performed on FFPE sections of cancer tissues on the Leica Biosystems BOND Rx automated staining instrument. Detection of the target epitope by primary antibody was amplified using the RNAscope® HiPlex12 Reagents Kit v2 in the T1 channel and detected using an HRP-conjugated label probe and DAB chromogen from the Leica BOND Polymer Refine Detection Kit. In one example, both single-labelled antibodies and higher-labelled antibodies with higher oligonucleotide labelling resulted in successful detection of KI67 in cervical cancer tissue, with minimal background observed with higher labeling (FIG. 5A). In another example, both single-labelled antibodies and higher-labelled antibodies with higher oligonucleotide labelling resulted in successful detection of CD8 in ovarian cancer tissue, with minimal background observed with higher labeling (FIG. 5B). In another example, both single-labelled antibodies and higher-labelled antibodies with higher oligonucleotide labelling resulted in successful detection of INSR in liver cancer tissue (FIG. 5C).

Example 3

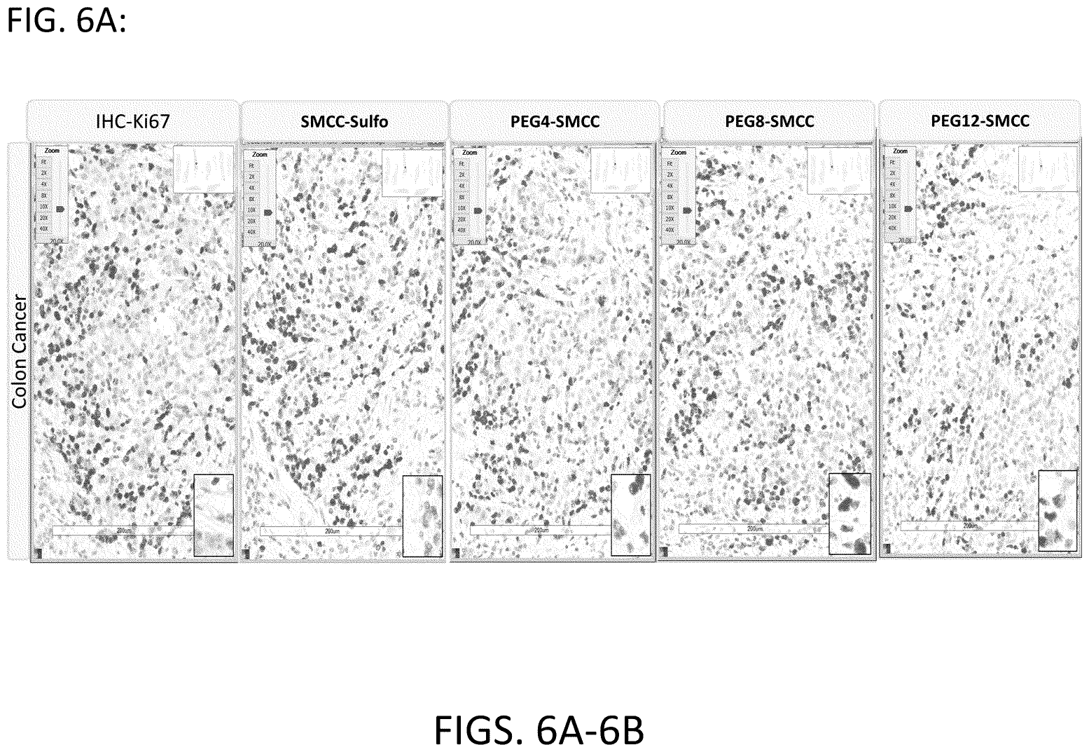

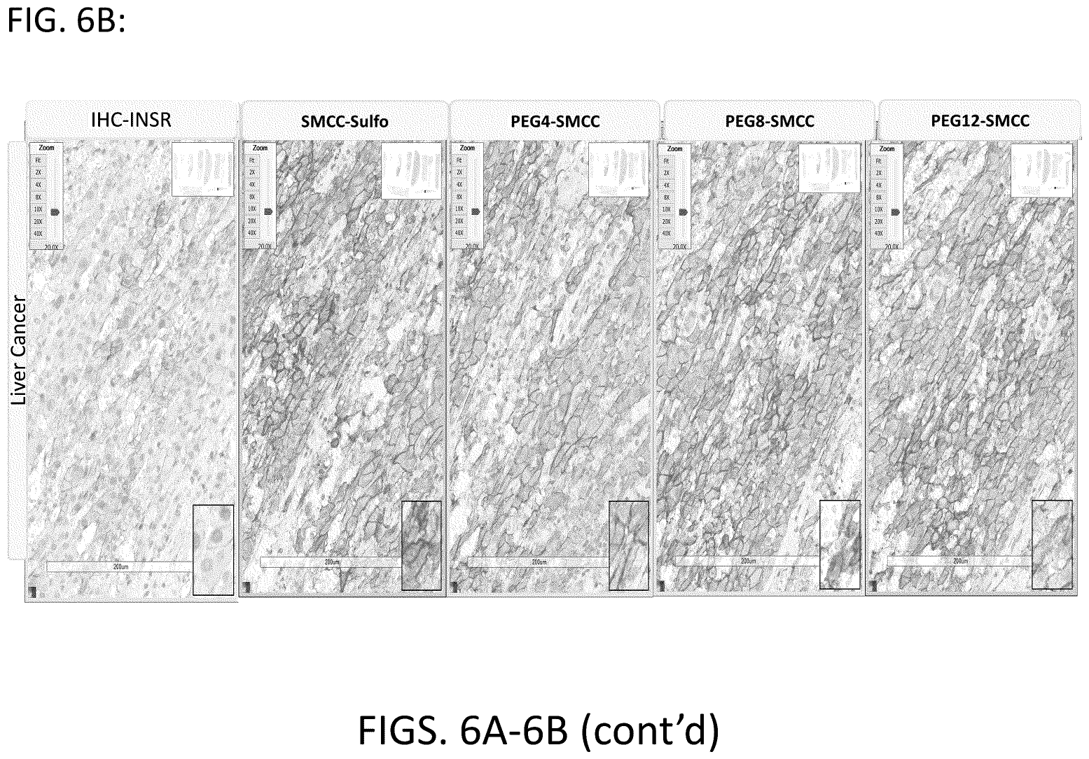

Experiments were also conducted to evaluate whether the use of a linker between the oligonucleotide sequence and antibody would allow for greater accessibility of the conjugated oligonucleotide sequence by the signal generating complex. Oligonucleotides with no linker, and with PEG4, PEG8, and PEG12 linkers, were conjugated to primary antibodies-of-interest. ProteinScope staining was performed on FFPE sections of cancer tissues and compared to IHC on distinct sections of the same cancer tissue samples on the Leica Biosystems BOND Rx automated staining instrument. Detection of the target epitope by primary antibody was amplified using the RNAscope® HiPlex12 Reagents Kit v2 in the T1 channel and detected using an HRP-conjugated label probe and DAB chromogen from the Leica BOND Polymer Refine Detection Kit. In one example, primary antibody with direct oligonucleotide labelling and antibody-oligonucleotide conjugate with PEG4, PEG8, and PEG12 linkers demonstrated successful detection of KI67 in colon cancer tissue (FIG. 6A). In another example, primary antibody with direct oligonucleotide labelling and antibody-oligonucleotide conjugate with PEG4, PEG8, and PEG12 linkers demonstrated successful detection of INSR in liver cancer tissue (FIG. 6B).

Example 4

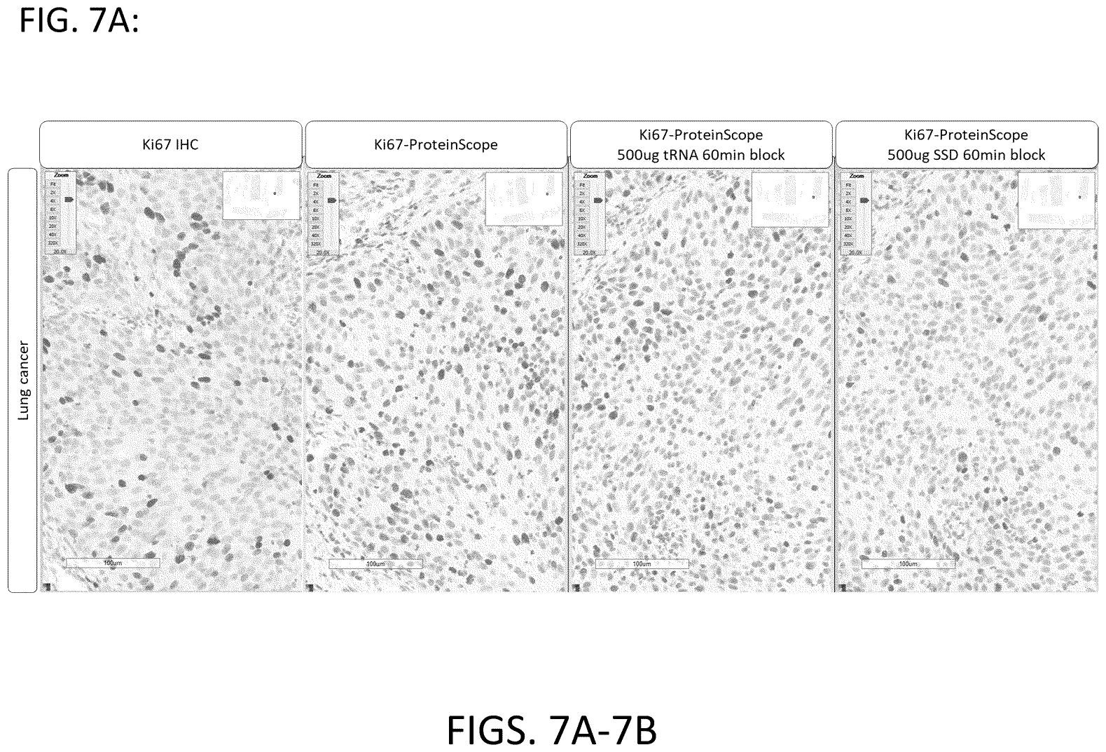

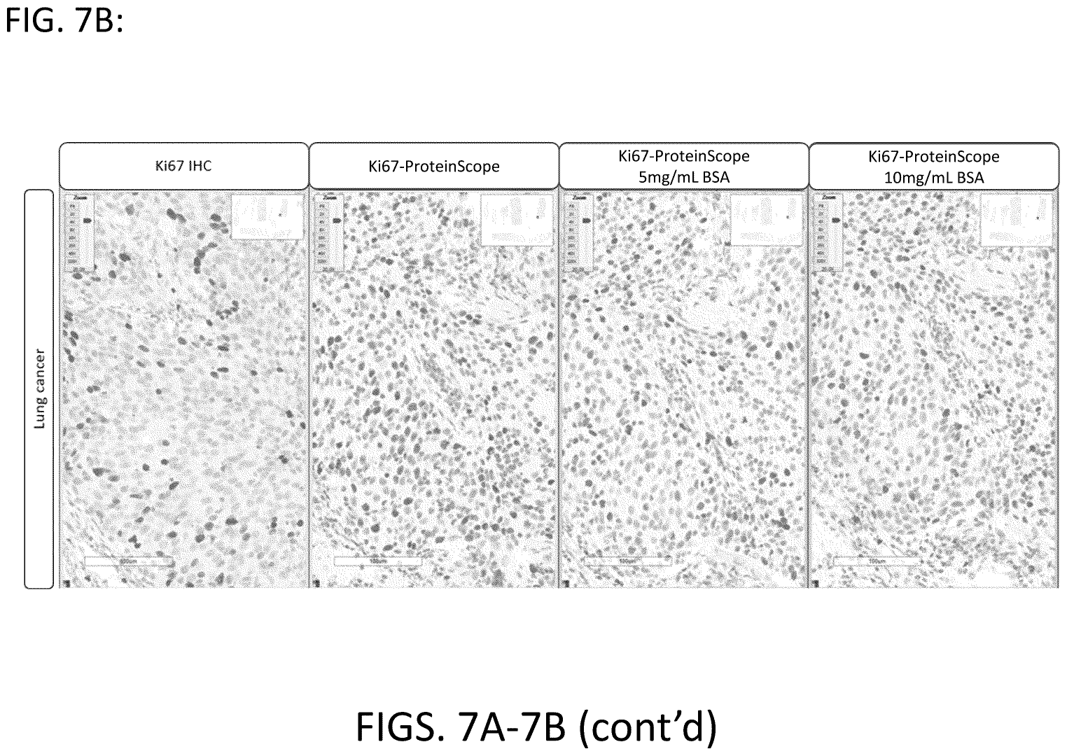

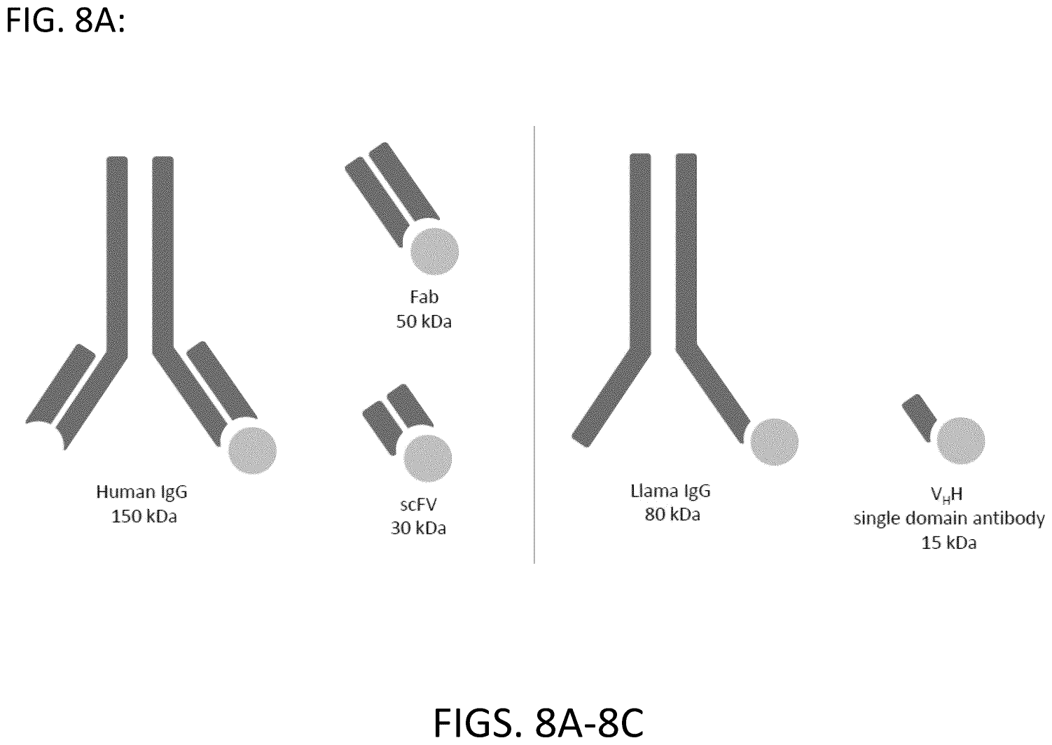

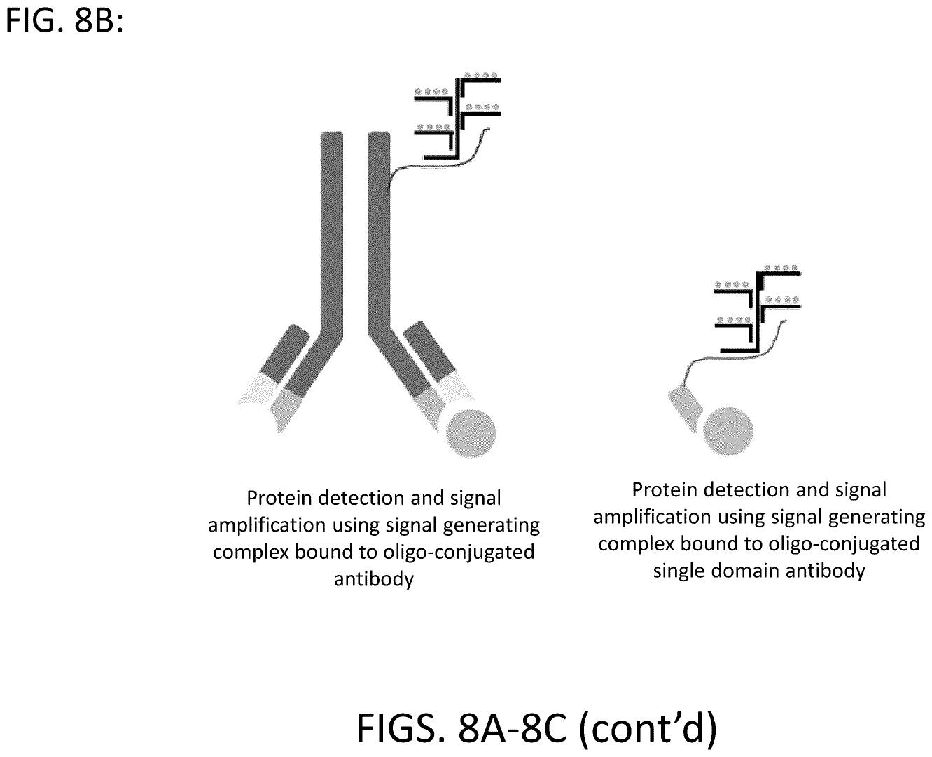

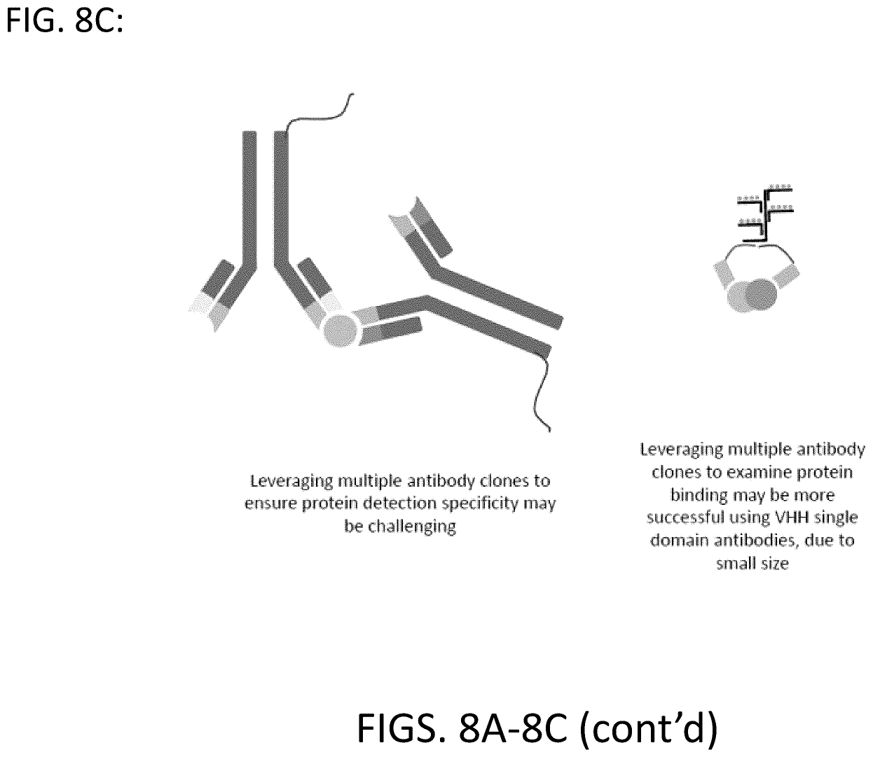

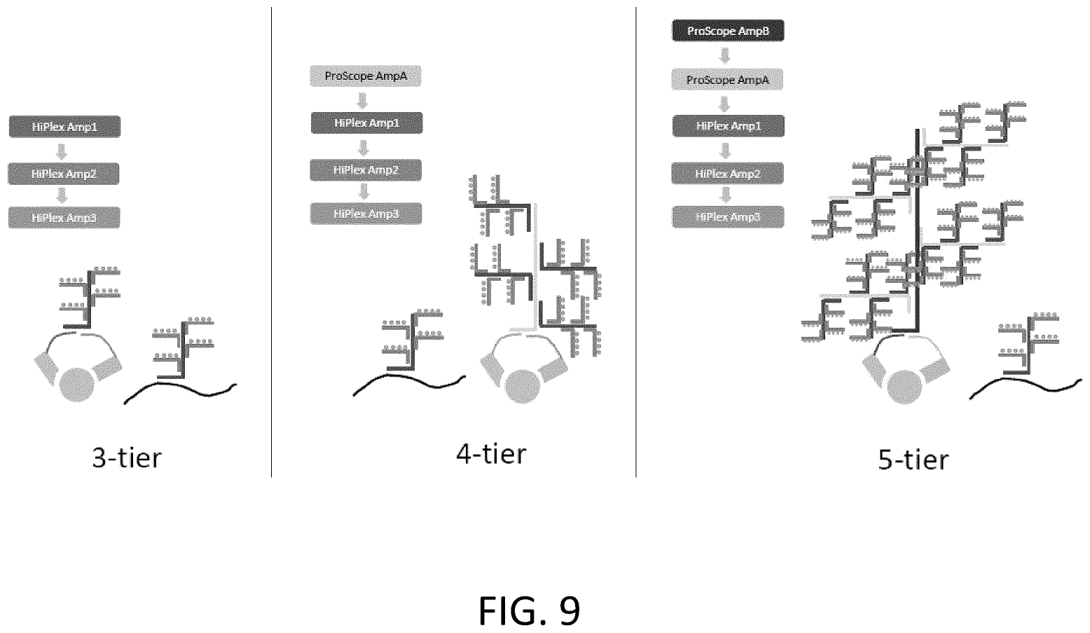

Experiments were also conducted to test whether the inclusion of blocking reagents in the ProteinScope workflow may help reduce background from various sources. For example, ProteinScope performed using antibody-oligonucleotide conjugates with higher degree of labeling (DOL) produced somewhat higher background levels compared to ProteinScope performed using antibodies with single oligonucleotide labeling and the traditional IHC baseline (FIGS. 5A-5B). However, this somewhat elevated level of background staining was reduced by multiple blocking strategies (FIG. 7). In one example, KI67 signal was maintained and background was reduced with addition of a 60 min blocking step with 500 ug/ml tRNA prior to primary antibody incubation, as compared to ProteinScope performed without pre-primary blocking (FIG. 7A). In another example, KI67 signal was maintained and background was reduced with addition of a 60 min blocking step with 500 ug/ml Salmon Sperm DNA prior to primary antibody incubation, as compared to ProteinScope performed without pre-primary blocking (FIG. 7B). In another example, KI67 staining was maintained and background was reduced using primary antibody concentrate diluted in a Co-Detection Antibody Diluent with the addition of 5 mg/ml BSA, as compared to ProteinScope staining using KI67 primary antibody concentrate diluted in Co-Detection Antibody Diluent alone (FIG. 7B). KI67 staining was maintained and background was reduced using primary antibody concentrate diluted in a Co-Detection Antibody Diluent with the addition of 10 mg/m1 BSA, as compared to ProteinScope staining using KI67 primary antibody concentrate diluted in Co-Detection Antibody Diluent alone (FIG. 7B).

Example 5

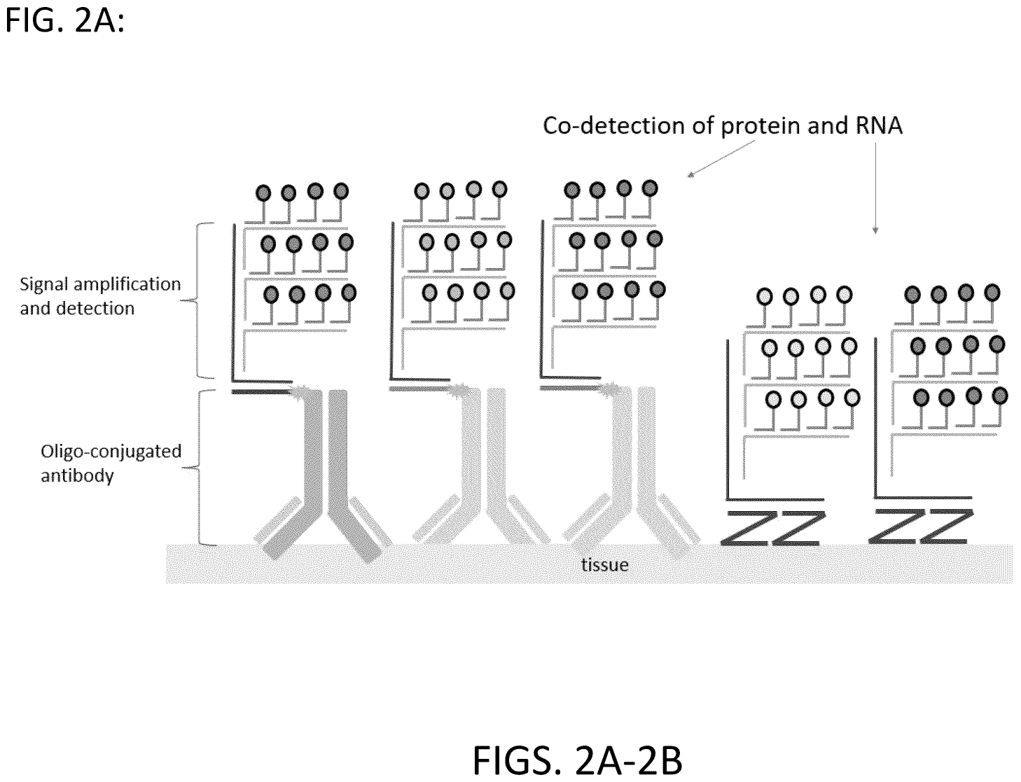

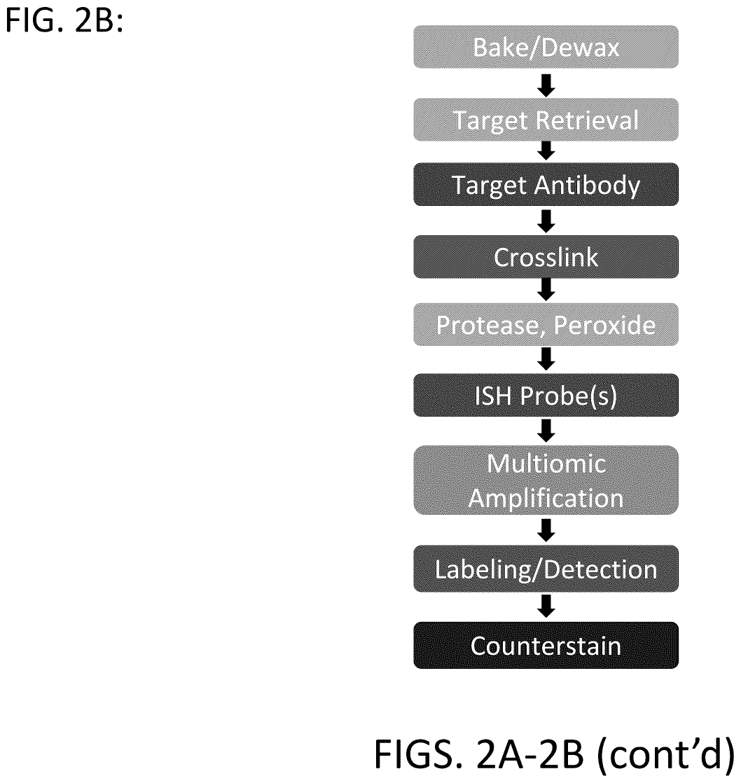

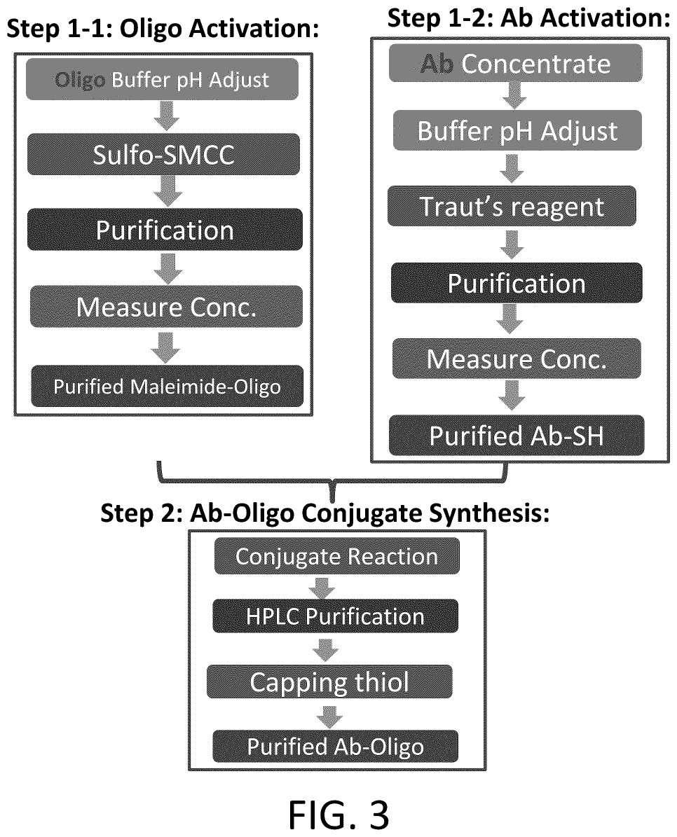

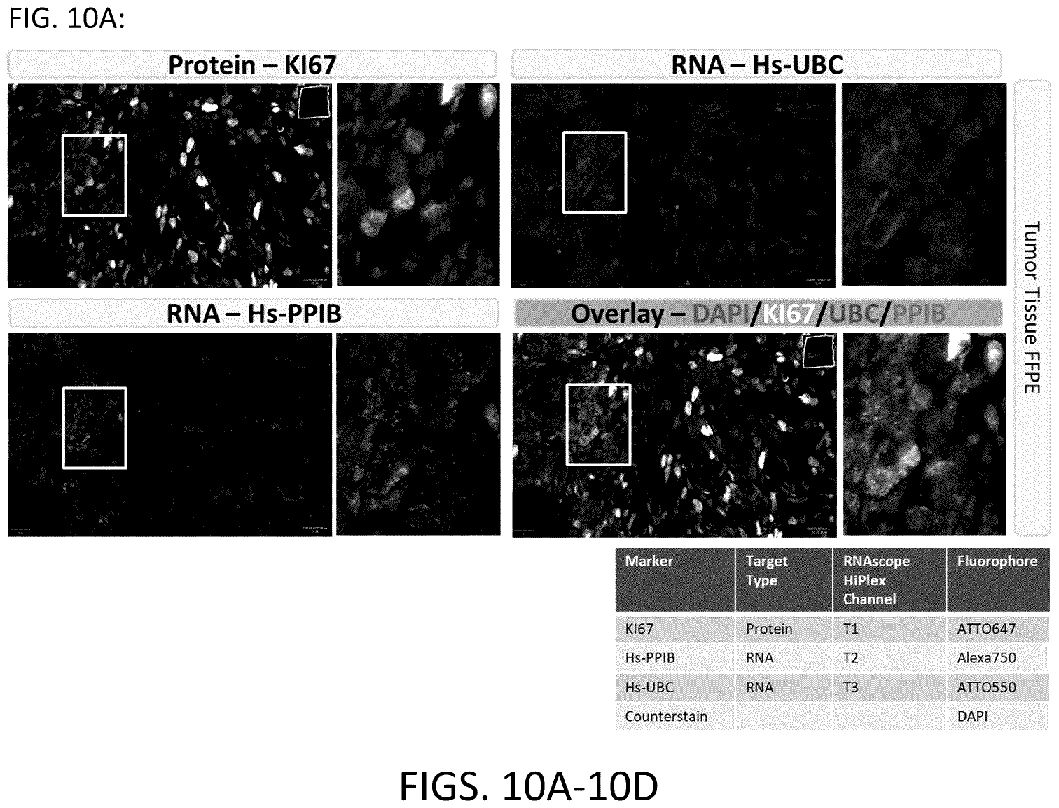

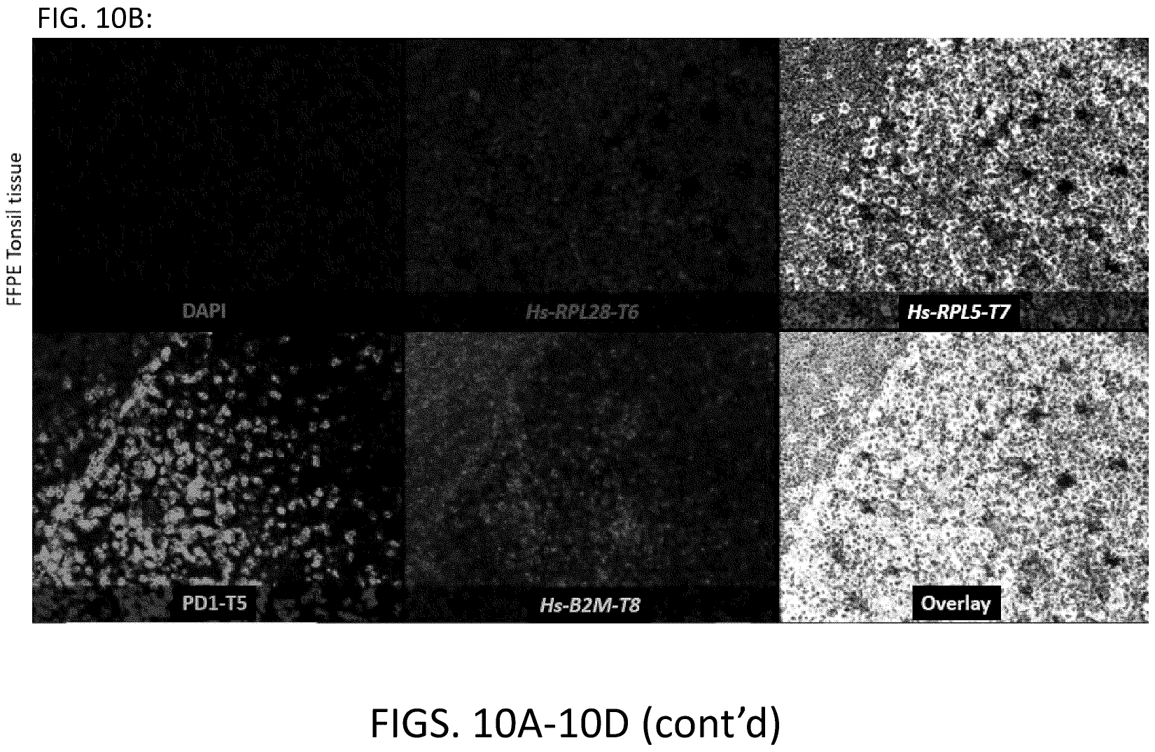

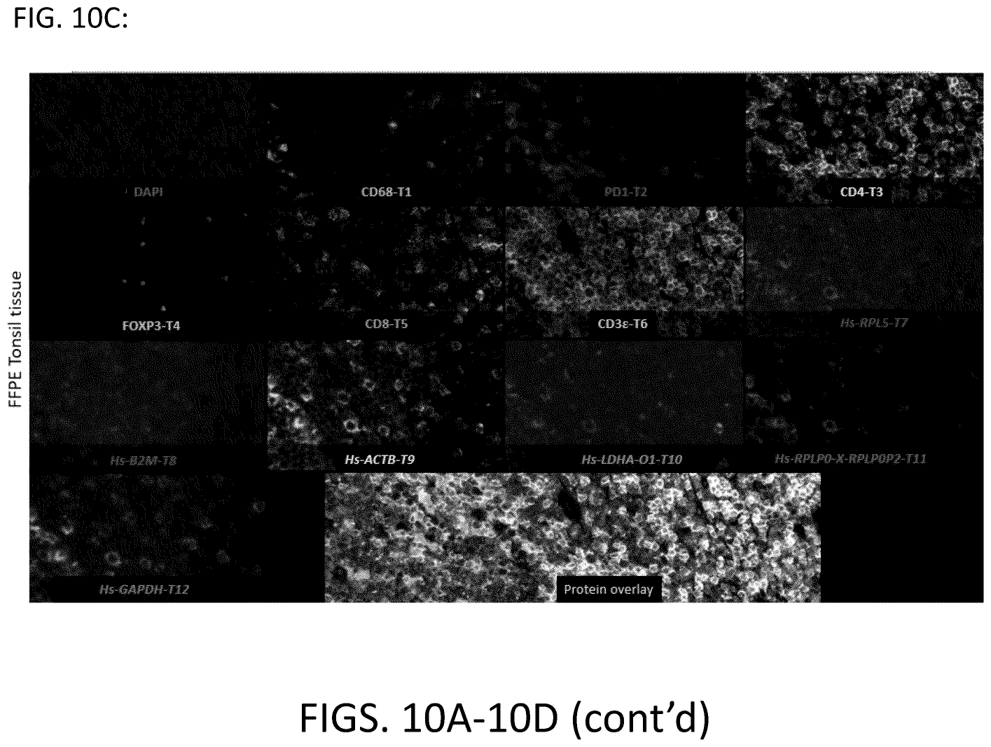

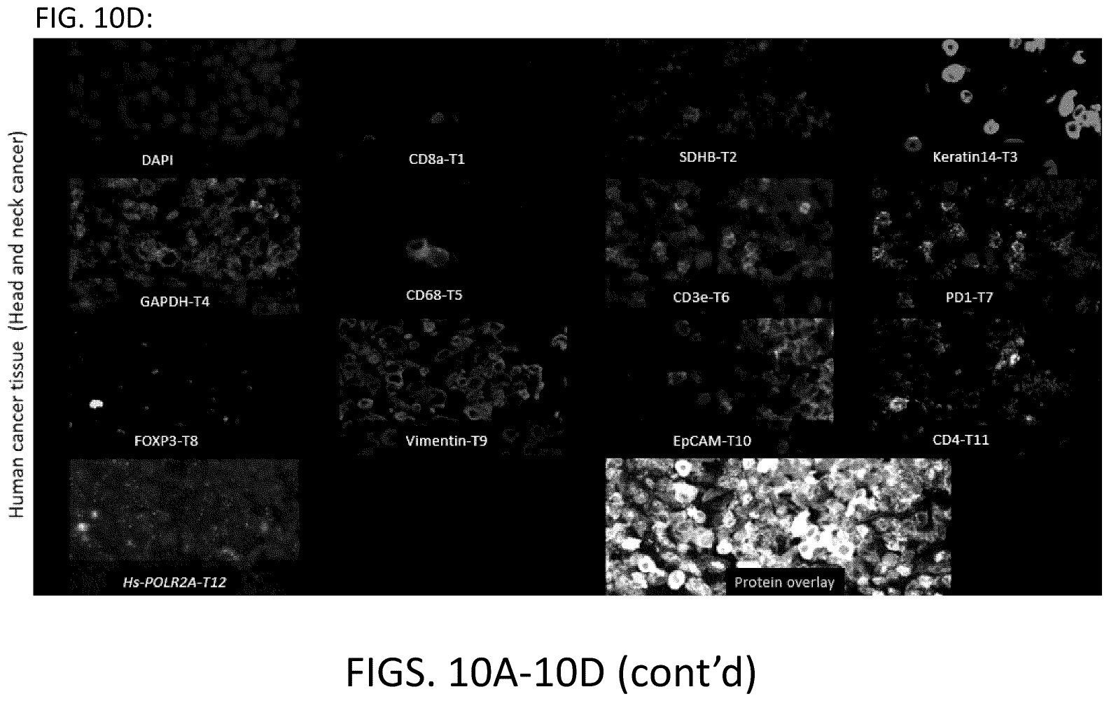

As described further herein, ProteinScope allows for the simultaneous detection of target protein and target nucleic acids in the same FFPE tissue section (i.e., multiplexed, multi-omic visualization) with spatial resolution at the single cell level using a single workflow (FIGS. 2A-2B). In this exemplary workflow, ProteinScope for RNA and protein co-detection can be automated on the Leica Biosystems BOND Rx automated staining instrument, using primary antibody conjugated to a T1 channel oligonucleotide using Sulfo-SMCC crosslinking. Antibody-bound oligonucleotide can be crosslinked with 10% neutral buffered formalin prior to sample treatment with protease and peroxide reagents and hybridization of a cocktail of RNAscope® HiPlex probes of Hs-PPIB-T2 and Hs-UBC-T3. Signal generating complexes are formed simultaneously for T1, T2, and T3 channels, encompassing both protein and RNA detection, using the RNAscope® HiPlex12 Reagents Kit v2, with T1 channel detected by ATT0647, T2 channel by Alexa750, and T3 by ATT0550, followed by counterstaining with DAPI. Using this workflow, KI67 protein was co-detected with Hs-PPIB and Hs-UBC protein in the same tissue section (FIG. 10).

Additionally, FIG. 10B includes representative images demonstrating multiplex staining using the ProteinScope workflow showing one protein marker (PD1) in T5 channel (green) and three mRNA markers in T6 (red), T7 (white) and T8 (pink) channels on FFPE human tonsil tissue. The mRNA markers are shown in italics. FIG. 10C includes representative images demonstrating multiplex staining using the ProteinScope workflow showing six protein markers (CD68, PD1, CD4, FOXP3, CD8, CD3e) and six mRNA markers (shown in italics) on FFPE human tonsil tissue. Bottom panel shows overlay of protein markers only. FIG. 10D includes representative images demonstrating multiplex staining using the ProteinScope workflow showing eleven protein markers (CD8a, SDHB, Keratin-14, GAPDH, CD68, CD3e, PD1, FOXP3, Vimentin, EpCAM, CD4) and one mRNA marker (Hs-POLR2A, shown in italics) on FFPE human head and neck cancer tissue. Bottom right panel shows overlay of protein markers only.

Taken together, these data clearly demonstrate the effective multiplexed detection of both protein and nucleic acid targets using the compositions and methods of the present disclosure.

Example 6

Experiments were conducted to develop the compositions and methods for target detection using oligo-conjugated secondary antibodies (FIG. 12A). Traditional IHC does not allow for simultaneous detection of antibodies from the same host due to cross-reactivity of secondary antibody with the different primary antibodies. However, amplification of oligo through signal-generating complex enables host-independent protein detection. For example, any number of primary antibodies from the same or multiple hosts can be individually complexed with oligo-conjugated respective secondary antibodies in individual tubes (FIGS. 12B and 12C). In the same tubes, any unbound sites in the primary-secondary antibody complex are then quenched using serum against the host species. The conjugated oligo on the secondary antibody is then used for signal amplification using a signal-generating complex, allowing for simultaneous amplification and detection of multiple proteins (FIGS. 12B and 12C). Shown above is an example with dual detection of two primary antibodies from rabbit host (FIGS. 12B and 12C).

FIG. 12D includes representative images demonstrating similar staining between the ProteinScope workflow utilizing oligo-conjugated antibodies and traditional immunohistochemistry utilizing secondary antibody-based detection. FIG. 12D includes representative images demonstrating dual protein staining (right panel) with anti-rabbit secondary antibody conjugated to T1 and T3 channel oligos. CD3e and PD1 primary antibodies from rabbit host are mixed with oligo-conjugated anti-rabbit secondary antibodies in individual tubes, quenched with rabbit serum, and then functionally tested using the ProteinScope workflow either in singleplex staining (left and middle panel) or as a cocktail (right panel). FIG. 12E includes representative images demonstrating triple protein staining for three rabbit anti-human antibodies, GFAP, NeuN and IBA1, detected using oligo-conjugated secondary antibodies in the ProteinScope workflow. Corresponding immunofluorescence controls are shown in individual panels.

Example 7

Experiments were also conducted to determine the effects of glutaraldehyde as a cross-linking reagent to improve protein detection sensitivity. For example, simultaneous detection of protein and mRNA requires the use of protease for optimal RNA detection, which negatively impacts protein staining. To counteract this, it has been previously shown that introducing a post-primary antibody cross-linking step using 10% neutral buffered formalin (NBF) provides adequate protection of antibody-antigen binding from subsequent protease degradation. In order to further improve the sensitivity of protein detection, other cross-linking reagents were investigated, including Glyoxal and Glutaraldehyde. Of the tested reagents, glutaraldehyde showed significant improvement in protein detection sensitivity compared to 10% NBF. However, addition of glutaraldehyde resulted in high autofluorescence background, hindering RNA signal interpretation. Diluting glutaraldehyde in 10% NBF enabled reduction in glutaraldehyde-alone induced autofluorescence, while also retaining higher protein staining sensitivity than 10% NBF alone. This has been demonstrated at various concentrations including 1%, 0.5% and 0.1% glutaraldehyde in 10% NBF.

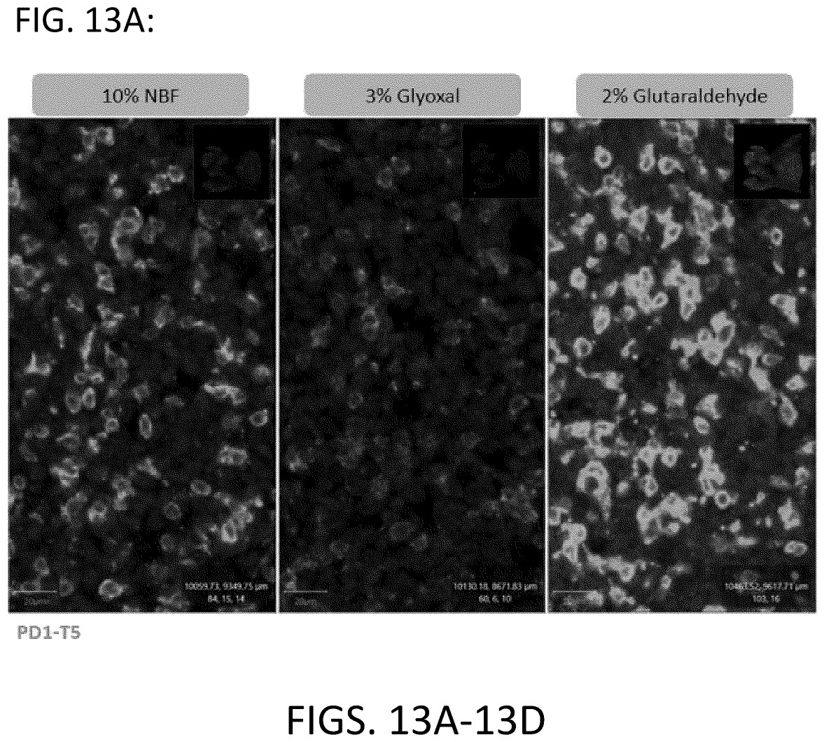

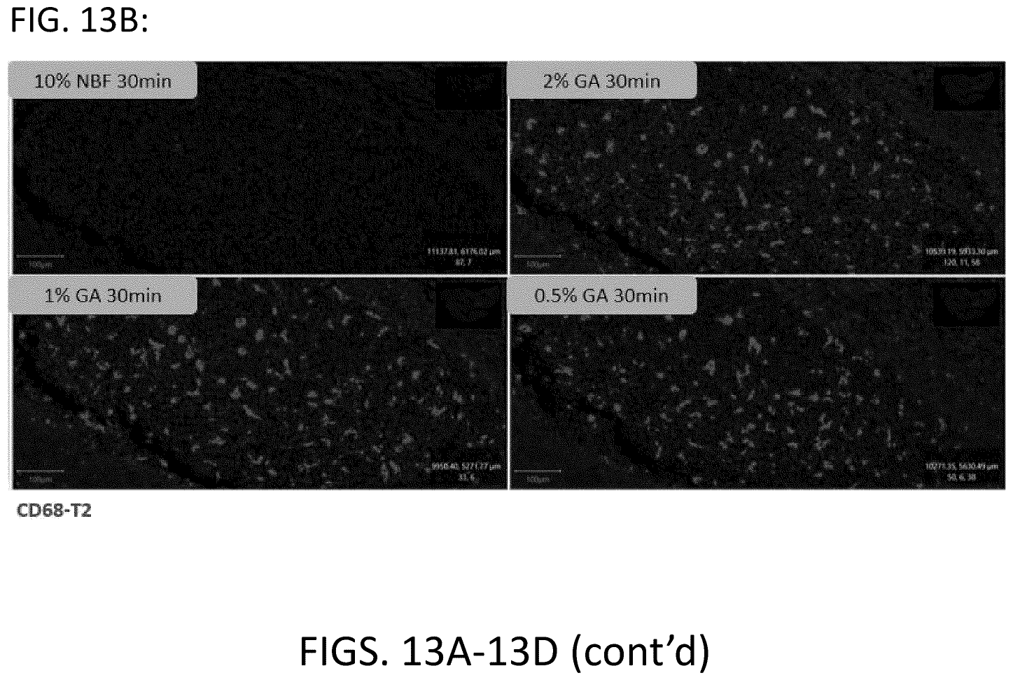

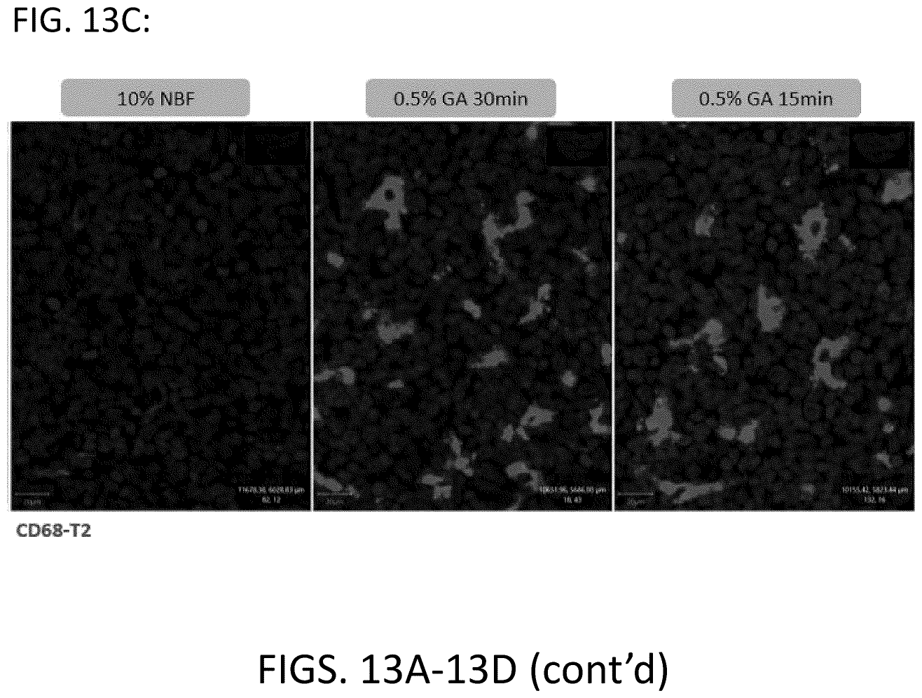

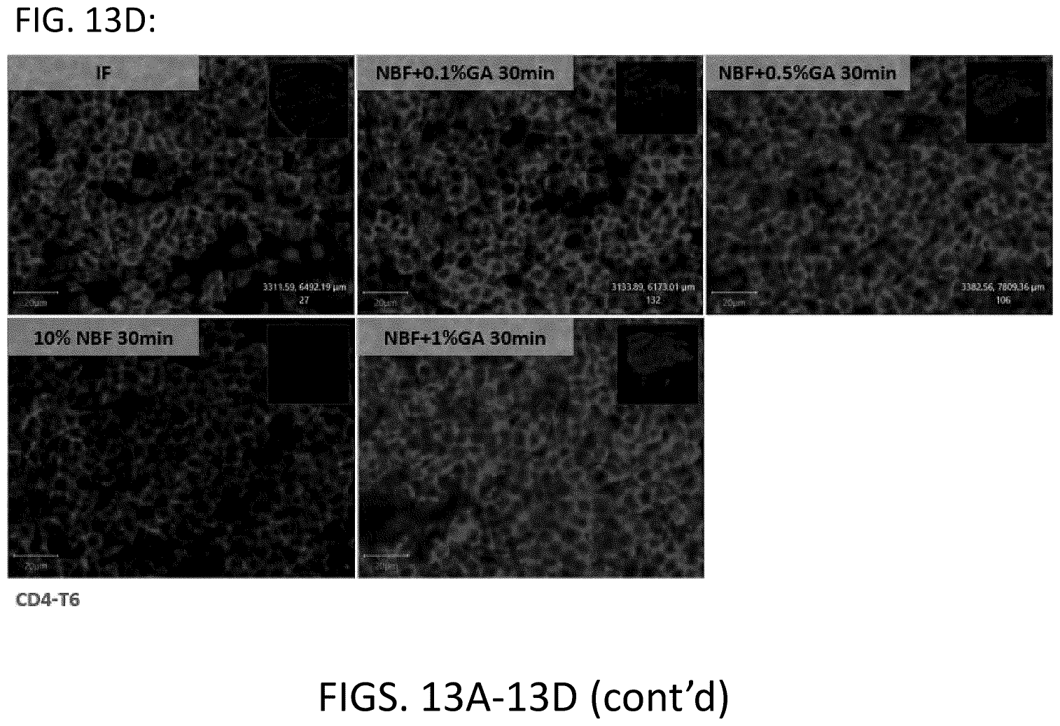

As shown in FIG. 13A, PD1 protein signal detection (channel T5) was stronger with glutaraldehyde cross-linking after the primary antibody step. Glutaraldehyde cross-linking also enhanced detection of more PD1 positive cells than 10% NBF. The data from FIG. 13B demonstrated that protein signal detection for CD68 marker was stronger than control (10% NBF) at all concentrations of glutaraldehyde tested (GA = glutaraldehyde). FIG. 13C demonstrated that shorter cross-linking time with glutaraldehyde retains increased sensitivity compared to control (10% NBF). Additionally, the data from FIG. 13D demonstrated that dilution of glutaraldehyde in 10% NBF reduces autofluorescence. Post-antibody cross-linking using different concentrations of glutaraldehyde mixed in 10% NBF demonstrated better sensitivity of CD4 detection than control (10% NBF).

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A method for detecting a target protein in a fixed biological sample, the method comprising:
(i) contacting the fixed biological sample with at least one secondary antibody or a fragment thereof, wherein the at least one secondary antibody or the fragment thereof is covalently attached to an oligonucleotide;
(ii) contacting the fixed biological sample with a signal-generating complex, wherein the signal-generating complex comprises a nucleic acid component capable of hybridizing to the oligonucleotide; and
(iii) detecting a signal from the signal-generating complex.

2. The method of claim 1, wherein the at least one secondary antibody or fragment thereof is specific for at least one antigen on at least one primary antibody, and wherein the at least one primary antibody is specific for at least one antigen on a target protein.

3. The method of claim 2, wherein the at least one secondary antibody or fragment thereof comprises at least two secondary antibodies specific for different antigens on the same primary antibody.

4. The method of claim 2, wherein the at least one secondary antibody or fragment thereof comprises at least two secondary antibodies specific for the same antigen on a primary antibody.

5. The method of claim 2, wherein the at least one secondary antibody or fragment thereof comprises at least one secondary antibody specific for an antigen on a primary antibody, and at least one additional secondary antibody specific for an antigen on at least one additional primary antibody.

6. The method of claim 5, wherein the primary antibody and the at least one additional primary antibody are (i) specific for the same antigen on a target protein, or (ii) specific for different antigens on different target proteins.

7. The method of claim 1, wherein the at least one secondary antibody or fragment thereof is selected from a monoclonal antibody, a multispecific antibody, a bispecific antibody, a trispecific antibody, a tetravalent antibody, a single-domain antibody, and a chimeric antibody.

8. The method of claim 1, wherein the at least one secondary antibody or fragment thereof is selected from a Fab, a scFv, a Fv, a scFv-Fc, a Fab′, a Fab′-SH, a F(ab′)₂, a diabody, a minibody, a tribody, a nanobody, and an affibody.

9. The method of claim 1, wherein the oligonucleotide is covalently attached to the at least one secondary antibody via a linker.

10. The method of claim 9, wherein the linker comprises at least one of 6-hydrazinonicotinate (HyNic), succinimidyl-4-(N-maleimidomethyl)cyclohexane-1-carboxylate (SMCC), and/or polyethylene glycol (PEG).

11. The method of claim 9, wherein the linker comprises at least one reactive moiety.

12. The method of claim 11, wherein the at least one reactive moiety comprises at least one of a succinimidyl ester, a sulfosuccinimidyl ester, a pentafluorophenyl ester, a maleimide, an azide, an alkyne, a hydrazine, an isocyanate, an isothiocyanate, and/or a haloacetamide.

13. The method of claim 9, wherein the linker comprises an oligonucleotide sequence.

14. The method of claim 1, wherein the method further comprises contacting the fixed biological sample with a blocking agent comprising at least one of tRNA, salmon sperm DNA, herring DNA, calf thymus DNA, bovine serum albumin, casein, normal goat serum, normal swine serum, normal chicken serum, and/or fish serum.

15. The method of claim 1, wherein the method further comprises contacting the fixed biological sample with a crosslinking agent comprising at least one of formalin, bis(succinimidyl) polyethylene glycol, and/or glutaraldehyde.

16. The method of claim 1, wherein the signal-generating complex comprises at least one amplifier.

17. The method of claim 1, wherein the signal-generating complex comprises at least one detectable label.

18. The method of claim 17, wherein the at least one detectable label comprises a fluorescent moiety or a chromogenic moiety.

19. The method of claim 1, wherein the fixed biological sample is a tissue sample or is derived from a tissue sample; a blood sample or is derived from a blood sample; a cytological sample or is derived from a cytological sample; a sample comprising cultured cells; or a sample comprising exosomes.

20. The method of claim 1, wherein the fixed biological sample is a fixed tissue sample.

21. The method of claim 1, wherein the fixed biological sample is a formalin-fixed paraffin-embedded (FFPE) tissue sample.

22. A kit for detecting a target protein in a fixed biological sample, comprising:
(i) at least one secondary antibody or a fragment thereof, wherein the at least one secondary antibody or the fragment thereof is covalently attached to an oligonucleotide; and
(ii) a signal-generating complex, wherein the signal-generating complex comprises a nucleic acid component capable of hybridizing to the oligonucleotide.

23. The kit of claim 22, wherein the kit further comprises a control or reference sample.
